(19) United States
(12) Patent Application Publication
(10) Pub. No.: US 2023/0001396 A1
(43) Pub. Date: Jan. 5, 2023

(54) COMPOSITIONS AND METHODS FOR CONTROLLED DELIVERY AND PROTECTION OF THERAPEUTIC AGENTS

(71) Applicant: Board of Regents, The University of Texas System, Austin, TX (US)

(72) Inventors: Jeremiah J. GASSENSMITH, Allen, TX (US); Michael LUZURIAGA, Dallas, TX (US); Ronald SMALDONE, Plano, TX (US)

(73) Assignee: Board of Regents, The University of Texas System, Austin, TX (US)

(21) Appl. No.: 17/776,803

(22) PCT Filed: Nov. 13, 2020
(86) PCT No.: PCT/US2020/060392

§ 371 Date: May 13, 2022

Related U.S. Application Data
(60) Provisional application No. 62/935,401, filed on Nov. 14, 2019.

(51) Int. Cl. B01J 31/12; A61K 31/765; A61K 9/00; A61K 35/76

(52) U.S. Cl. CPC B01J 31/123; A61K 31/765; A61K 9/0024; A61K 9/0021; A61K 35/76

(57) ABSTRACT

In some aspects, the present disclosure provides pharmaceutical compositions comprising a) a therapeutic agent; b) a metal-organic framework (MOF) or a coordination polymer; and c) a pharmaceutically acceptable polymer; wherein the therapeutic agent is encapsulated within the metal-organic framework or coordination polymer to form an encapsulated therapeutic agent, and wherein the encapsulated therapeutic agent is further encapsulated, entrapped, embedded, dispersed within, or complexed to the pharmaceutically acceptable polymer. The present disclosure also provides methods of making said compositions, methods of treating a disease or disorder comprising administering to a subject said compositions. The present disclosure also provides microneedles and implantable medical devices comprising said compositions.

### PRIORITY CLAIM

This application claims benefit of priority to U.S. Provisional Application 62/935,401, filed Nov. 14, 2019, the entire contents of which are hereby incorporated by reference.

### BACKGROUND

### I. Field

The present disclosure relates generally to the fields of chemistry and materials science. More particularly, it concerns compositions comprising therapeutic agents encapsulated in nanoporous materials and further encapsulated, entrapped, embedded, dispersed within, or complexed to pharmaceutically acceptable polymers. Also described herein are methods for stabilizing, protecting, and delivering therapeutic agents to a subject in need thereof.

### II. Description of Related Art

Proteinaceous therapeutics are moving to the forefront of medicine for their specificity in treatments, favorable side effect profiles, and their potential in personalized medicine (Leader et al., 2008; Chen et al., 2016). Unfortunately, many of these proteins are structurally metastable (Thirumalai et al., 2011) and they can undergo drastic conformational changes at elevated temperatures, in organic solvents, and at pHs different from physiological conditions (Mallamace et al., 2016; Carmichael et al., 2015). These problems limit proteins to short-term low-temperature storage that require costly infrastructure in place to keep them stable throughout shipping. Researchers have been motivated by these limitations and have begun to develop new methods that can enhance protein stability (Pisal et al., 2006; LeClair et al., 2018; Sridhar et al., 2018; Welch et al., 2018; Lee et al., 2017; Liu et al., 2017; Vrdoljak et al., 2016).

Metal-organic framework (MOF) encapsulation has been shown (Doonan et al., 2017) to stabilize enzymes (Alsaiari et al., 2018; Liang et al., 2015), viruses (Li et al., 2016; Li et al., 2018), and antibodies (Wang et al., 2016) while providing structural and chemical protection. MOFs are highly porous crystalline materials made of metal-ion clusters linked by organic ligand struts (Rosi et al., 2002; McGuire et al., 2015) that have shown promise for use in gas storage (Banerjee et al., 2008) and separation (Hayashi et al., 2007; Li et al., 2018), catalysis (Huxley et al., 2018; Otake et al., 2018), sensing (Fan et al., 2018), and small molecule drug delivery (Zhuang et al., 2014; Adhikari et al., 2015; Zheng et al., 2016; Laźaro et al., 2018). Recently, researchers have shown that MOFs can immobilize (Majewski et al., 2017; Ricco et al., 2018) and stabilize biomacromolecules (Nadar et al., 2018; Li et al., 2016). In particular, coating proteins in zeolitic imidazolate framework-8 (ZIF-8) is proving to be a promising method for protection against conditions normally adverse to proteins, and there have been many promising advancements in this area (Nadar et al., 2018; Hoop et al., 2018; Maddigan et al., 2018; Liao et al., 2017; Wang et al., 2018). In particular, biomimetic mineralized growth (Liang et al., 2015; Li et al., 2016; Ricco et al., 2018) of ZIF-8 directly onto the surface of a protein has emerged as a means to encapsulate enzymes and insulin using only protein, zinc salts, and methylimidazole directly in water (Wang et al., 2018; Zhu et al., 2018). Because ZIF-8 can grow on protein surfaces of different sizes, charge states, and morphologies, this process is quite “agnostic” to the biomolecule host inside the ZIF (Li et al., 2018; Maddigan et al., 2018). This differs from other equally elegant methods that use bespoke MOFs with tuned (Deng et al., 2012; Chen et al., 2018; Li et al., 2016) pore sizes to encapsulate specific biomolecules or polymer-encapsulated proteins coated with ZIF and formed in organic protic solvents (Zhang et al., 2016; Lyu et al., 2014).

For instance, the inventors' lab biomimetically encapsulated tobacco mosaic virus (TMV) within a ZIF-8 shell (TMV@ZIF) and found the encapsulation process to be high yielding and promoted by a modest affinity for zinc ions toward the proteinaceous surface (Li et al., 2018). This affinity leads to high local concentrations of zinc, which promotes a mineralization process that results in either core—shell or monolithic crystals of bionanoparticles (Liang et al., 2015; Xu et al., 2007). It is unclear, however, how or if the nucleation and growth affects the secondary or tertiary structure at the protein surface. If the protein surface of a therapeutic protein is altered as a result of the nucleation process, unwanted immunological reactions may occur as human proteins would not be recognized as “self” and antigens for encapsulated vaccines would raise antibodies against a misfolded protein rendering this strategy moot (Xu et al., 2016; Maurer et al., 2005; Stephanopoulos et al., 2011). Furthermore, a variety of therapeutic agents exhibit limitations, such as thermal instability. As such, there exists a need for compositions and methods for the delivery of therapeutic agents having improved properties.

### SUMMARY

In some aspects, the present disclosure provides pharmaceutical compositions comprising a) a therapeutic agent; b) a metal-organic framework (MOF) or a coordination polymer; and c) a pharmaceutically acceptable polymer; wherein the therapeutic agent is encapsulated within the metal-organic framework or coordination polymer to form an encapsulated therapeutic agent, and wherein the encapsulated therapeutic agent is further encapsulated, entrapped, embedded, dispersed within, or complexed to the pharmaceutically acceptable polymer. In some embodiments, the metal-organic framework or coordination polymer comprises zirconium, iron, or zinc. In some embodiments, the composition comprises a coordination polymer. In some embodiments, the composition comprises a MOF. In further embodiments, the metal-organic framework is a zeolitic imidazolate framework (ZIF), such as ZIF-8.

In some embodiments, the therapeutic agent is a vaccine. In some embodiments, the therapeutic agent is a small molecule, a peptide or polypeptide, or a nucleotide or polynucleotide. In some embodiments, the therapeutic agent is a small molecule. In some embodiments, the small molecule is an antibiotic or a chemotherapeutic. In some embodiments, the therapeutic agent is a protein or a nucleic acid. In some embodiments, the therapeutic agent is derived from bacterial, protozoal, or microbial origin. In some embodiments, the therapeutic agent is a virus, a virus-like particle (VLP), a bacterium, or a bacterium-like particle (BLP). In some embodiments, the therapeutic agent is a virus. In some embodiments, the vaccine is an inactivated vaccine or a live-attenuated vaccine. In some embodiments, the therapeutic agent elicits an immune response.

In some embodiments, the pharmaceutically acceptable polymer is polylactic acid. In some embodiments, the pharmaceutically acceptable polymer is polycaprolactone. In some embodiments, the pharmaceutically acceptable polymer is a co-polymer. In some embodiments, the co-polymer is a block co-polymer. In some embodiments, the pharmaceutically acceptable polymer is poly(lactic-co-glycolic acid). In some embodiments, the pharmaceutically acceptable polymer is a blend of polymers. In some embodiments, the blend comprises polylactic acid, polycaprolactone, or poly(lactic-co-glycolic acid). In some embodiments, the blend comprises polylactic acid, polycaprolactone, and poly(lactic-co-glycolic acid).

In some embodiments, the composition is formulated as a colloid. In some embodiments, the composition is formulated for injection.

In another aspect, the present disclosure provides implantable medical devices comprising a composition of the present disclosure. In some embodiments, the composition is comprised within a thin-film. In some embodiments, the thin-film is present on the surface of the device.

In still another aspect, the present disclosure provides microneedles comprising a composition of the present disclosure. In some embodiments, the microneedle is coated with the composition. In some embodiments, the microneedle consists essentially of the composition. In some embodiments, the microneedle is attached to an adhesive patch.

In yet another aspect, the present disclosure provides methods of treating and/or preventing a disease or disorder in a subject in need thereof comprising administering to the subject an effective amount of a composition of the present disclosure. In some embodiments, the composition comprises a vaccine.

In another aspect, the present disclosure provides methods of making a composition of the present disclosure comprising contacting a therapeutic agent with a MOF and a pharmaceutically acceptable polymer. In some embodiments, the method is performed in a single reaction vessel. In some embodiments, the method further comprises a solvent. In some embodiments, the solvent is water. In some embodiments, the solvent is an aqueous solution comprising at least 50% water by volume.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one of these drawings in combination with the detailed description of specific embodiments presented herein.

FIG. 1 shows schematic for analyzing surface effects from encapsulation and stressing: TMV contains glutamate residues on the interior pore modifiable with EDC chemistry; the viral RNA is embedded inside the TMV pore; (a) native TMV is incubated with 2-methylimidazole and zinc acetate to form TMV@ZIF; (b) TMV@ZIF is subjected to denaturing conditions such as heat and organic solvents; (c) stressed TMV@ZIF is exfoliated with EDTA; (d) recovered TMV surface integrity is characterized by ELISA.

FIG. 2 shows SEM images (top panel) of TMV@ZIF (a) non-stressed, (b) heating at 100° C. for 20 min, and after soaking overnight in (c) methanol, (d) 6 M guanidinium chloride, and (e) ethyl acetate. Scale bars represent 1 μm. Top panel (f) shows TEM image of exfoliated non-stressed TMV. Scale bar is 200 nm. Bottom panel shows the ELISA response of naked and encapsulated TMV subject to no stress (a), heating (b), methanol (c), 6 M guanidinium chloride (d), and ethyl acetate (e). These labels correlate to the SEM images a—e of the top panel. The percentages range from buffer blank (0% TMV) to a separate internal control of non-stressed naked TMV (100% TMV).

FIG. 3 shows N. benthamiana plants (top panel) 10 days after inoculation with (a) 0.1 M pH 7.4 potassium phosphate buffer as a negative control, (b) TMV@ZIF, (c) exfoliated TMV@ZIF, and (d) native TMV as a positive control. Bottom panel shows a bar graph showing the viral recovery of TMV from 1 g of harvested leaves measured by ELISA. Leaves were inoculated with buffer as a negative control, TMV@ZIF, exfoliated TMV@ZIF, and native TMV as a positive control.

FIGS. 4A-4C show time schedule showing the days the mice were injected (bottom arrows) and submandibular blood withdrawals were performed (top arrows) (FIG. 4A). Serum samples were diluted 200×, 1000×, and 5000×. FIG. 4B shows ELISA response for each time point, buffer blank subtracted. FIG. 4C shows hematoxylin & eosin Y (H&E) staining of saline- and TMV@ZIF-injected mice.

FIGS. 5A-5C show fluorescence intensity over time (FIG. 5A). The baseline is the average fluorescence intensity of four mice before injection. The dashed line represents the error of the baseline. FIG. 5B shows images of the mice prior to injection of Cy5-TMV or Cy5-TMV@ZIF. The mice were shaved and the only initial fluorescence comes from the hairs on the head. FIG. 5C shows after injection and time point images of Cy5-TMV or Cy5-TMV@ZIF.

FIG. 6 shows excitation and emission spectra of Cy5-TMV. Excitation λₘₐₓ=647 nm; emission λₘₐₓ=666 nm.

FIG. 7 shows regions of interest and their and their radiant efficiencies (×107): 1) Mouse skin: 21.2, 2) Saline: 2.7, 3) Cy5-TMV: 386.3, 4) Cy5-TMV@ZIF: 104.3, and 5) ZIF-8 in water: 3.6. It should be noted that the quantity of Cy5-TMV in tubes 3 and 4 are the same, however, the ZIF shell attenuates the fluorescence.

FIG. 8 shows UV-Vis absorbance at 646 nm of Cy5-COOH in solution and Cy5-TMV.

FIG. 9 shows PXRD spectra of stressed TMV@ZIF samples.

FIG. 10 shows ELISA response of test mice after 10 days.

FIGS. 11A & 11B show extended release method. FIG. 11A shows ZIF protects antigens and slowly releases them in vivo. FIG. 11B shows ZIF imbedded into a degradable polymer can largely prevent antigen release for at least 10 days until polymer shell is dissolved, then release will start. Simultaneous administration of antigen@ZIF@polymer would provide ˜20 days of sustained release.

FIG. 12 shows schematic diagram of PLGA encapsulated ZIF-8 nanoparticle preparation steps.

FIGS. 13A & 13B show scanning electron microscopy (SEM) images of ZIF-8 (500 nm).

FIGS. 14A & 14B show SEM images of Cy5@ZIF-8.

FIGS. 15A & 15B show SEM images of ZIF-8@PLGA microparticles.

FIGS. 16A & 16B show SEM images after treatment of PLGA microspheres with chloroform and butanol.

FIGS. 17A & 17B show fluorescence spectra of Cy5@ZIF@PLGA in water (FIG. 17A) and methanol (FIG. 17B).

FIGS. 18A & 18B show PXRD spectra of encapsulated Cy5 dye. FIG. 18A shows PXRD of Cy5@ZIF-8@PLGA microparticles. FIG. 18B shows a comparison between the observed PXRD pattern for Cy5@ZIF-8 and the calculated PXRD for ZIF-8.

FIGS. 19A-19D show fluorescence spectra of Cy5@ZIF-8@PLGA in phosphate-buffered saline (PBS) and Cy5 in PBS. FIGS. 19A and 19B show the fluorescence spectrum of Cy5@ZIF-8@PLGA in PBS at pH 7.4 and 5.4, respectively. FIGS. 19C and 19D show the fluorescence spectrum of Cy5 in PBS at pH 7.4 and 5.4, respectively.

FIGS. 20A & 20B shows dynamic light scattering (DLS) spectra of Cy5@ZIF-8 (FIG. 20A) and Cy5@ZIF-8@PLGA (FIG. 20B).

FIG. 21 shows schematic diagram of PLGA encapsulated smURFP@ZIF-8 nanoparticle preparation steps.

FIGS. 22A & 22B show SEM images of smURFP@ZIF-8 (1 mg/mL).

FIGS. 23A & 23B show SEM images of smURFP@ZIF-8 (0.2 mg/mL).

FIGS. 24A & 24B show SEM images of smURFP@ZIF-8 (0.3 mg/mL).

FIGS. 25A & 25B show SEM images of smURFP@ZIF-8@PLGA microparticles.

FIG. 26 shows DLS spectrum of smURFP@ZIF-8.

FIG. 27 shows fluorescence spectrum of smURFP@ZIF@PLGA. The maximum emission at 670 nm after excitation at 642 nm confirms the presence of smURF protein within the microparticles.

FIGS. 28A & 28B show gel electrophoresis results of exfoliating smURFP@ZIF-8 particles. smURFP@ZIF-8 particles were treated with exfoliation solution (0.5 M EDTA solution 600 pH 7.9) and then run through 1 Agarose gel to observe the presence of protein inside ZIF-8 molecule and subsequently the plate was imaged with Cy5 channel with Typhoon (FIG. 28A) or stained with Coomassie blue dye (FIG. 28B). From left to right native smURFP (0.25 mg/mL), 1 mg/mL smURFP@ZIF-8, 0.2 mg/mL smURFP@ZIF-8 and 0.3 mg/mL smURFP@ZIF-8 after exfoliation.

FIG. 29 shows PXRD pattern of ZIF-8 and smURFP@ZIF-8. Similarity of XRD patterns suggest that encapsulation of smURFP does not change the structural integrity of ZIF-8. Powdered XRD data was taken with smart Rigaku XRD machine from 5 to 45 degrees, speed 3.

### DESCRIPTION OF ILLUSTRATIVE EMBODIMENTS

The present disclosure provides compositions for improved delivery of therapeutic agents. In some aspects, the compositions comprise a therapeutic agent encapsulated within a metal-organic framework or coordinate polymer and further encapsulated within a pharmaceutically acceptable polymer. Also provided herein are methods of treating or preventing disease using the compositions of the present disclosure.

### I. Compositions of the Present Disclosure

All the compositions of the present invention may in some embodiments be used for the prevention and treatment of one or more diseases or disorders discussed herein or otherwise. In some embodiments, one or more of the compositions characterized or exemplified herein as an intermediate, a metabolite, and/or prodrug, may nevertheless also be useful for the prevention and treatment of one or more diseases or disorders. As such unless explicitly stated to the contrary, all the compositions of the present invention are deemed “active compositions” and “therapeutic compositions” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compositions of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compositions known in the prior art, whether for use in the indications stated herein or otherwise.

In addition, atoms making up the compounds that make up the compositions of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

### II. Therapeutic Agents

In some aspects, the present disclosure provides compositions comprising a therapeutic agent. The “therapeutic agent” used in the present methods and compositions refers to any substance, compound, drug, medicament, or other primary active ingredient that provides a therapeutic, diagnostic, prophylactic, and/or pharmacological effect when administered to a subject, such as a human Non-limiting examples of therapeutic agents include small molecules, peptides or polypeptides, or nucleotides or polynucleotides, antibiotics, chemotherapeutics, vaccines, or a compound that elicits an immune response. Further non-limiting therapeutic agents include proteins or nucleic acids. In some embodiments, the therapeutic agent may be derived from bacterial, protozoal, or microbial origin. In some embodiments, the therapeutic agent is a virus, a virus-like particle (VLP), a bacterium, or a bacterium-like particle (BLP).

Further non-limiting examples of suitable therapeutic agents include anticancer agents, antifungal agents, psychiatric agents such as analgesics, consciousness level-altering agents such as anesthetic agents or hypnotics, nonsteroidal anti-inflammatory drugs (NSAIDS), anthelminthics, antiacne agents, antiallergic agents, antianginal agents, antiarrhythmic agents, anti-asthma agents, antibacterial agents, anti-benign prostate hypertrophy agents, antibiotics agents, anticoagulants, anticonvulsants, antidepressants, antidiabetics, antiemetics, antiepileptics, antigout agents, antihypertensive agents, antiinfective agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitumoral agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, hematopoietics, histamine receptor antagonists, hormones (e.g., steroid hormones), immunosuppressants, keratolytics, lipid regulating agents, leukotriene inhibitors, macrolides, muscle relaxants, neuroleptics, nutritional agents, opioid analgesics, protease inhibitors, sedatives, or vasodilators.

Non-limiting examples of the therapeutic agents may include 7-Methoxypteridine, 7 Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In some embodiments, the therapeutic agent is a peptide or a polypeptide. In some embodiments, the polypetide is an antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In some embodiments, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies that may be comprised in the compositions provided herein include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex ((17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. Nos. 5,736,167, 7,060,808, and 5,821,337, which are incorporated by reference herein.

Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-β antibody); metelimumab/CAT-192 (anti-TGF-β antibody); lerdelimumab/CAT-152 (anti-TGF-β antibody); ID11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 β7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

Other exemplary polypeptides include, but not limited to insulin, insulin-like growth factor, human growth hormone (hGH), tissue plasminogen activator (tPA), cytokines, such as interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TNF-related apoptosis-inducing ligand (TRAIL); granulocyte colony-stimulating factor (G-CSF), granulocyte-macrophage colony-stimulating factor (GM-CSF), macrophage colony-stimulating factor (M-CSF), monocyte chemotactic protein-1 (MCP-1), vascular endothelial growth factor (VEGF), erythropoietin, or any other hormone growth factors. Additional suitable biologically active polypeptides include, but are not limited to, amylin, salmon-derived calcitonin (s-CT), glucagon-like peptide 1 (GLP-1), glucagon, parthyroid hormone (PTH1), oxytocin, desmopressin (8 D-Arg vasopressin), insulin, protein YY (PYY), cytokines and lymphokines such as IFNα, IFNβ, IFNγ.

While specific examples of the polypeptide for use in accordance with this disclosure are mentioned below, this does not mean that other known peptides or proteins are excluded. These peptides or proteins may be naturally occurring, recombinant or chemically synthesized substances.

The following is a partial listing of such peptides or proteins: cytokines, peptide hormones, growth factors, factors acting on the cardiovascular system, cell adhesion factors, factors acting on the central and peripheral nervous systems, factors acting on humoral electrolytes and hemal organic substances, factors acting on bone and skeleton, factors acting on the gastrointestinal system, factors acting on the kidney and urinary organs, factors acting on the connective tissue and skin, factors acting on the sense organs, factors acting on the immune system, factors acting on the respiratory system, factors acting on the genital organs, and various enzymes.

In some embodiments, the polypeptides are cytokines, peptide hormones, growth factors, factors acting on the cardiovascular system, factors acting on the central and peripheral nervous systems, factors acting on humoral electrolytes and hemal organic substances, factors acting on bone and skeleton, factors acting on the gastrointestinal system, factors acting on the immune system, factors acting on the respiratory system, factors acting on the genital organs, and enzymes. The cytokines include tymphokines, monokines, and hematopoietic factors. The lymphokines include interferons (e.g. interferon-α, -β and -γ), and interleukins (e.g. interleukin 2 through 11). The monokines include interleukin-1, tumor necrosis factors (e.g. TNF-α and -β), and malignant leukocyte inhibitory factor (LIF). The hematopoietic factors include, among others, erythropoietin, granulocyte colony stimulating factor (G-CSF), granulocyte-macrophage stimulating factor (GM-CSF) and macrophage colony stimulating factor (M-CSF). As factors having hematopoietic activity, factors having thrombopoietic (proliferation) activity, such as a leukocyte proliferation factor preparation (Leucoprol, Morinaga Milk), thrombopoietin, platelet proliferation stimulating factor and megakaryocyte proliferation (stimulating) factor could also be used.

The factors acting on bone and skeleton include bone GLa peptide, parathyroid hormone and its active fragments (osteostatin), histone H4-related bone formation and proliferation peptide (OGP) and their muteins, derivatives and analogs thereof.

The growth factors include nerve growth factors (NGF, NGF-2/NT-3), epidermal growth factor (EGF), fibroblast growth factor (FGF), insulin-like growth factor (IGF), transforming growth factor (TGF), platelet-derived cell growth factor (PDGF), and hepatocyte growth factor (HGF).

Peptide hormones include insulin, growth hormone, luteinizing hormone-releasing hormone (LH-RH), adrenocorticotropic hormone (ACTH), amylin, oxytocin, luteinizing hormone and other factors acting on the genital organs and their derivatives, analogs and congeners. As analogs of said LH-RH, such known substances are described in U.S. Pat. Nos. 4,008,209, 4,086,219, 4,124,577, 4,317,815 and 5,110,904, which are incorporated by reference herein.

The factors acting on the central and peripheral nervous systems include opioid peptides (e.g. enkepharins, endorphins, kyotorphins), neurotropic factor (NTF), calcitonin gene-related peptide (CGRP), thyroid hormone releasing hormone (TRH), and salts and derivatives of neurotensin.

### III. Nanoporous Materials

In some aspects, the present disclosure provides compositions comprising a nanoporous material, such as a metal-organic framework or a coordinate polymer. A nanoporous material is an organic or inorganic framework which contains a regular, porous structure having a pore size from about 0.2 to about 1000 nm. Within nanoporus materials, there are three major classifications of materials: microporous materials with a pore size from about 0.2 nm to about 2 nm, mesoporous materials with a pore size from about 2 nm to about 50 nm, or macroporous materials with a pore size from about 50 nm to about 1000 nm. In some embodiments, the present compositions relate to nanoporous materials which have a pore size from about 0.2 nm to about 100 nm, from about 1 nm to about 80 nm, or from about 5 nm to about 75 nm. The nanoporous material may have a pore size from about 1 nm, 2.5 nm, 5 nm, 10 nm, 15 nm, 20 nm, 25 nm, 30 nm, 35 nm, 40 nm, 45 nm, 50 nm, 60 nm, 70 nm, 80 nm, 90 nm, to about 100 nm, or any range derivable therein.

In some embodiments, the nanoporous material is a metal-organic framework. A metal-organic framework is a repeating metal ion or cluster with multiple organic ligands that form a porous higher dimension structure. Metal-organic framework may comprise a monovalent, a divalent, a trivalent, or a tetravalent ligand. Within these metal-organic frameworks exist pores which may be useful in absorbing another molecule such as a gas. In some embodiments, the metal-organic framework includes metal clusters that comprise a single metal ion, two metal ions, or three or more metal ions. The metal ion may be selected from the group consisting of Group 1 through 16 metals of the IUPAC Periodic Table of the Elements including actinides, and lanthanides, and combinations thereof. Non-limiting examples of suitable metal ions include Li⁺, Na⁺, K⁺, Rb⁺, Be²⁺, Mg²⁺, Ca²⁺, Sr²⁺, Ba²⁺, Sc³⁺, Y³⁺, Ti⁴⁺, Zr⁴⁺, Hf⁴⁺, V⁴⁺, V³⁺, V²⁺, Nb³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Ru²⁺, Os³⁺, Os²⁺, Co³⁺, Co²⁺, Rh²⁺, Rh⁺, Ir²⁺, Ir⁺, Ni²⁺, Ni⁺, Pd²⁺, Pd⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Tl³⁺, Si⁴⁺, Si²⁺, Ge⁴⁺, Ge²⁺, Sn⁴⁺, Sn²⁺, Pb⁴⁺, Pb²⁺, As⁵⁺, As³⁺, As⁺, Sb⁵⁺, Sb³⁺, Sb⁺, Bi⁵⁺, Bi³⁺, Bi⁺, and combinations thereof. Some non-limiting examples of metal organic frameworks include those taught by Kitagawa, et al., 2004, Ferey, 2008, and Furukawa, et al., 2013, all of which are incorporated in their entirety herein by reference. In some embodiments, the metal-organic framework is a zeolitic imidazolate framework, such as ZIF-8.

### IV. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a composition disclosed herein formulated with one or more excipients and/or drug carriers appropriate to the indicated route of administration. In some embodiments, the compositions disclosed herein are formulated in a manner amenable for the treatment of human and/or veterinary patients. In some embodiments, formulation comprises admixing or combining one or more of the compositions disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compositions may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compositions disclosed herein may be further coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compositions disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic agent in the compositions and preparations may, of course, be varied. The amount of the compositions in such pharmaceutical formulations is such that a suitable dosage will be obtained.

The therapeutic compositions may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal

In some embodiments, the effective dose range for the therapeutic compositions can be extrapolated from effective doses determined in animal studies for a variety of different animals In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a composition of the present disclosure or formulation comprising a composition of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks therebetween. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### V. Definitions

“Metal-organic frameworks” (MOFs) are framework materials, typically three-dimensional, self-assembled by the coordination of metal ions with organic linkers exhibiting porosity, typically established by gas adsorption. The MOFs discussed and disclosed herein are at times simply identified by their repeat unit as defined below without brackets or the subscript n. A mixed-metal-organic frameworks (M′MOF) is a subset of MOFs having two of more types of metal ions.

The term “unit cell” is basic and least volume consuming repeating structure of a solid. The unit cell is described by its angles between the edges (α, β, γ) and the length of these edges (a, b, c). As a result, the unit cell is the simplest way to describe a single crystal X-ray diffraction pattern.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂]ₙ—, the repeat unit is —CH₂CH₂—. The subscript “n” denotes the degree of polymerization, that is, the number of repeat units linked together. When the value for “n” is left undefined, it simply designates repetition of the formula within the brackets as well as the polymeric and/or framework nature of the material. The concept of a repeat unit applies equally to where the connectivity between the repeat units extends into three dimensions, such as in metal organic frameworks, cross-linked polymers, thermosetting polymers, etc. Note that for MOFs the repeat unit may also be shown without the subscript n.

“Pores” or “micropores” in the context of metal-organic frameworks are defined as open space within the MOFs.

“Multimodal size distribution” is defined as pore size distribution in three dimensions.

“Multidentate organic linker” is defined as ligand having several binding sites for the coordination to one or more metal ions.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Additionally, it is contemplated that one or more of the metal atoms may be replaced by another isotope of that metal. For example, the zinc atoms can be ⁶⁴Zn, ⁶⁶Zn, ⁶⁷Zn, ⁶⁸Zn, or ⁷⁰Zn. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “a□ido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, the formula

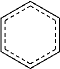

covers, for example,

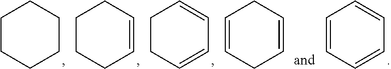

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

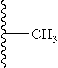

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

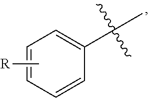

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

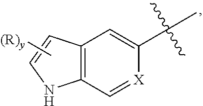

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n”' defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system.

An aromatic compound or chemical group may be depicted as a single resonance structure; however, depiction of one resonance structure is taken to also refer to any other resonance structure. For example:

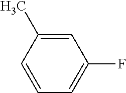

is also taken to refer to

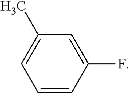

Aromatic compounds may also be depicted using a circle to represent the delocalized nature of the electrons in the fully conjugated cyclic π system, two non-limiting examples of which are shown below:

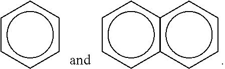

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

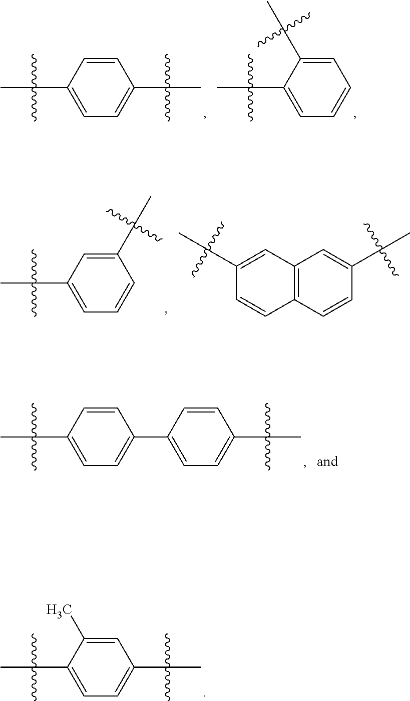

An “arene” refers to the class of compounds having the formula H—R, wherein R is aryl as that term is defined above. Benzene and toluene are non-limiting examples of arenes. The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the composition which, when administered to the patient or subject, is sufficient to effect such treatment or prevention of the disease as those terms are defined below.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis (3-hydroxy-2-ene-1-carboxylic acid), 4-methylbicyclo [2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent.

Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. Examples

The following examples are included to demonstrate preferred embodiments of the disclosure. It should be appreciated by those of skill in the art that the techniques disclosed in the examples which follow represent techniques discovered to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—TMV@ZIF-8 Nanoparticles

Example 1 may be further understood in view of Luzuriaga et al., 2019 and its associated supplemental materials, which are incorporated by reference herein.

### A. Introduction

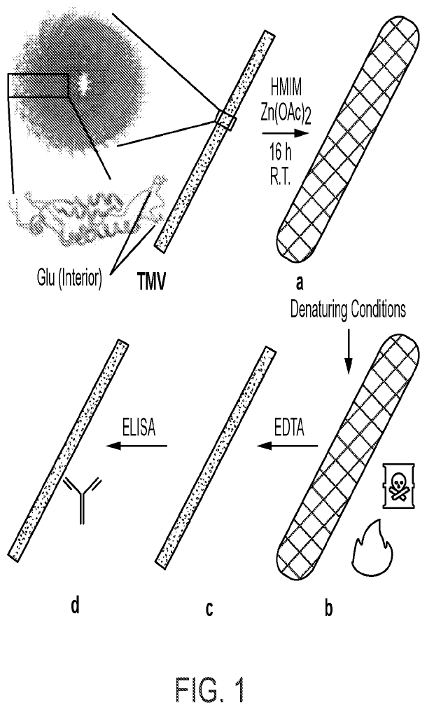

The inventors chose TMV, a 300 nm×18 nm tubular RNA plant virus, as a model vaccine biomacromolecule owing to extensive data on its in vivo performance as a carrier for engineered and chemically conjugated (Rybicki et al., 2014; Banik et al., 2015; Gasanova et al., 2016) epitopes in vaccine development (FIG. 1). This chemical modifiability, which can occur on both interior and exterior surfaces independently, has given TMV a unique appeal beyond vaccine development as the structure tolerates attachment of dyes (Masarapu et al., 2017), sensors (Dharmarwardana et al., 2018; Bäcker et al., 2017), contrast agents (Anderson et al., 2017), and bioactive molecules (Pitek et al., 2017; Finbloom et al., 2016). The multivalent nature of TMV comes from its 2130 identical coat proteins arranged helically around a 4 nm central pore where the viral RNA is located. This allows many bioconjugations to the same virus particle, increasing local concentration of active sites and immobilizing them, which is one reason it is thought to be such a useful platform for vaccine development (Rybicki et al., 2014; Banik et al., 2015; Gasanova et al., 2016).

Because TMV is an established preclinical vaccine platform, it is a reasonable model to test the efficacy of thermal protection when encapsulated inside ZIF-8. While it is possible to remove the ZIF-8 shell to obtain pristine TMV, the inventors wondered if this additional step was unnecessary. Indeed, it happens slowly in biological media, may present a method to formulate “slow release” agents for proteins an area of active research interest (Cosse et al., 2017; Ren et al., 2013). The inventors thus sought to determine if they could simply leave the TMV inside the protective ZIF-8 shell and inject this composite subcutaneously in a mouse model as a method to slowly release TMV, producing an immune response similar to injecting native TMV subcutaneously.

The inventors can quantify changes to the surfaces of TMV as a result of ZIF-8 growth and removal using anti-TMV antibodies measured in an enzyme-linked immunosorbent assay (ELISA). A damaged or unfolded protein at the virus surface will not interact strongly with their complementary antibodies and this loss of affinity will manifest as a diminished ELISA response. The TMV@ZIF composite was subjected to stressors, including heat and denaturing solvents, the ZIF shell was removed, and the recovered protein was examined by ELISA to confirm surface intactness. Tobacco plant infection and in vivo studies further demonstrate the viability of ZIF-8 as a protective shell. Finally, the inventors conducted longitudinal in vivo studies to ascertain the toxicity and immunogenicity of the TMV@ZIF-8 when implanted subcutaneously. These results show that this strategy has considerable potential to operate concurrently as a substrate to stabilize proteins at above ambient conditions as well as deliver them effectively intact and in a more linear dose.

### B. Results and Discussion

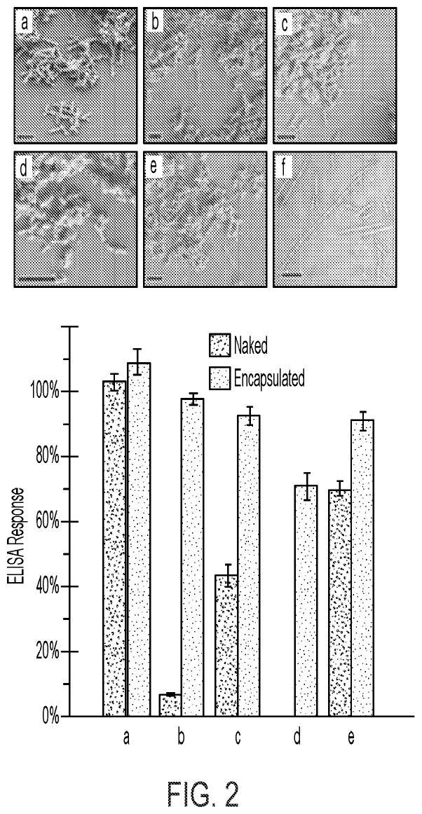

TMV@ZIF can be prepared in a number of different morphologies (Li et al., 2018) ranging from bulky rhombic dodecahedra containing hundreds of viruses to discrete rod-shaped core—shell bionanoparticles with a shell thickness tunable from 10 to 40 nm. Each of these morphologies have different colloidal and dispersion characteristics and for this study the following criteria were considered: (i) the composite made had to be dispersible in solution for easy in vivo injection, and (ii) the kinetics of shell dissolution should allow for complete dissolution of all in vivo administered ZIF-8 by the end of a 1-month study. When the inventors attempted to suspend rhombic dodecahedra, they settled out of solution too quickly and clogged the syringe. This is in line with literature reports that particles larger than 1000 nm tend to settle rather quickly, making them a difficult material for injection (Majewski et al., 2018; Chen et al., 2018). The inventors chose to continue forward with rods, as the ˜350 nm particle size allow for them to be easily dispersed into solution and the shell exfoliates more rapidly than the larger rhombic dodecahedra. They thus set out to determine whether the ZIF-8 shell would increase the stability of TMV and if it could be delivered in vivo. The encapsulation of TMV into ZIF-8 crystals was obtained by mixing TMV (0.111 mg) with an aqueous solution of 2-methylimidazole (400 mM, 3.0 mL), followed by an aqueous solution of zinc acetate (20 mM, 1.5 mL) (FIG. 1). After 16 h, the TMV@ZIF particles were collected by centrifugation at 4300 g and the shell diameter and rod-like morphology were verified by scanning electron microscopy (SEM) (FIG. 2, top panel, a). The morphology of TMV@ZIF is clearly different from the common rhombic dodecahedral native ZIF-8 crystals.

TMV@ZIF was then stressed under various conditions to determine the stability of the encapsulated virus surface. Stability versus various solvents was tested: soaking in methanol, ethyl acetate, and 6 M guanidinium chloride a common protein denaturant63 overnight. Thermal stabilitywas tested by heating TMV@ZIF to 100° C. for 20 mM. After stressing, samples retained their rodlike morphology, as seen in SEM (FIG. 2, top panel, b-e). The post-stressed composites were exfoliated in EDTA, desalted, and resuspended in 0.1 M sodium phosphate buffer. The protein concentrations were then determined by the Lowry assay, and all samples were diluted to 5.0×10⁻⁴ mg/mL and the ELISA response was determined. Because changes in the viral protein surface were being investigated, the ELISA results were normalized to naked non-stressed TMV (100%) and buffer blank (0%) for comparison between the two. The inventors were pleased to discover that the process of the shell formation and exfoliation did not significantly alter the protein surface and that the shell confers considerable protection to TMV when exposed to high temperatures. For instance, the percent difference between naked TMV and TMV@ZIF when heated to 100° C. for 20 min was 165.0% (FIG. 2, bottom, Table S1). Likewise, the percent difference between protected and unprotected exposure to the strongly denaturing guanidinium chloride was 199.2% (FIG. 1, bottom). The inventors were also able to demonstrate that the ZIF was able to confer protection against other denaturing organic solvents (Table 11).

TABLE 1
ELISA values of stressed TMV, stressed TMV@ZIF,
and their percent differences.

| | | | Percent |
|---|---|---|---|
| Stress | Naked | Encapsulated | Difference |
| Non-Stressed | 102.8 ± 2.3% | 108.9 ± 3.6% | 3.9% |
| Heated | 6.447 ± 0.188% | 97.53 ± 1.52% | 165.0% |
| Methanol | 43.02 ± 3.26% | 92.49 ± 2.63% | 55.42% |
| 6M Guanidine | 0.09033 ± 0.08277% | 70.70 ± 3.78% | 199.2% |
| HCl | | | |
| Ethyl Acetate | 69.72 ± 2.25% | 90.81 ± 2.84% | 18.33% |

The inventors then set out to determine whether encapsulating TMV would damage the RNA. To assess the protection that TMV@ ZIF has on the RNA of TMV, Nicotiana benthamiana plants were inoculated with phosphate buffer as a negative control and TMV@ZIF, TMV@ZIF exfoliated with EDTA, and native TMV as a positive control. The infection of N. benthamiana depends on the disassembly of the capsid to liberate the intact viral RNA and begin replication. Consequently, any damage to the RNA will reduce viral load in plants.

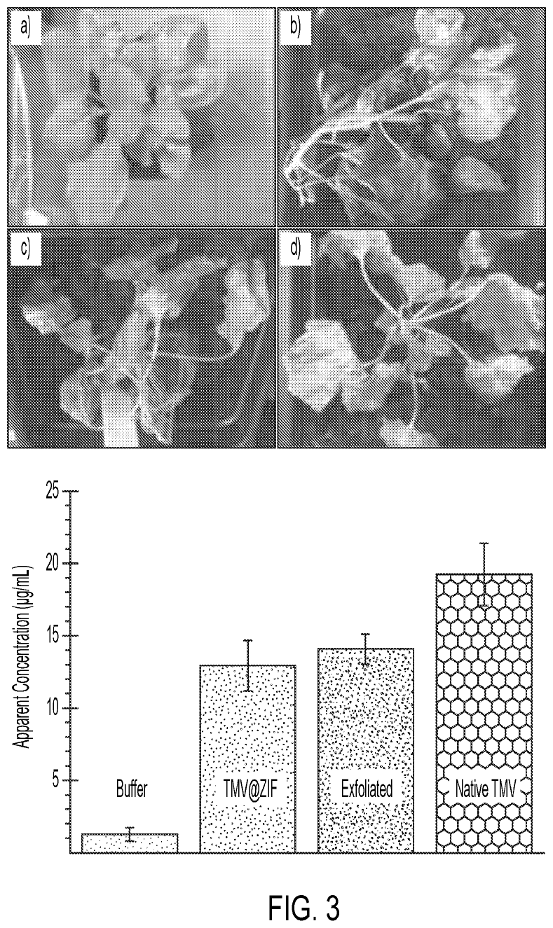

Inoculated leaves were collected after 10 days post infection. Visually, the control plants remained green and the other plants withered (FIG. 3, top panel). ELISA was performed on 1 g of leaves macerated in 10 mL of extraction buffer and centrifuged to remove the large plant matter. Because the relative amount of TMV present in the leaf matter was being investigated, the ELISA results were fit to a standard curve of native TMV and the results are reported as apparent TMV concentration in μg/mL. The TMV@ZIF, exfoliated TMV@ ZIF, and native TMV plants showed a clear increase in ELISA response compared to the buffer-inoculated plants, with percent differences of 164.32% (a 10-fold increase), 167.01% (an 11-fold increase), and 175.29% (a 15-fold increase), respectively (FIG. 3, bottom panel). This indicates that the TMV remains virulent and that the RNA survives the encapsulation and exfoliation process.

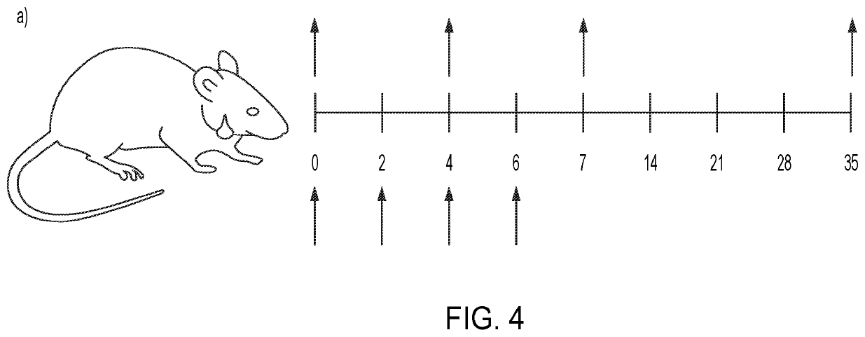

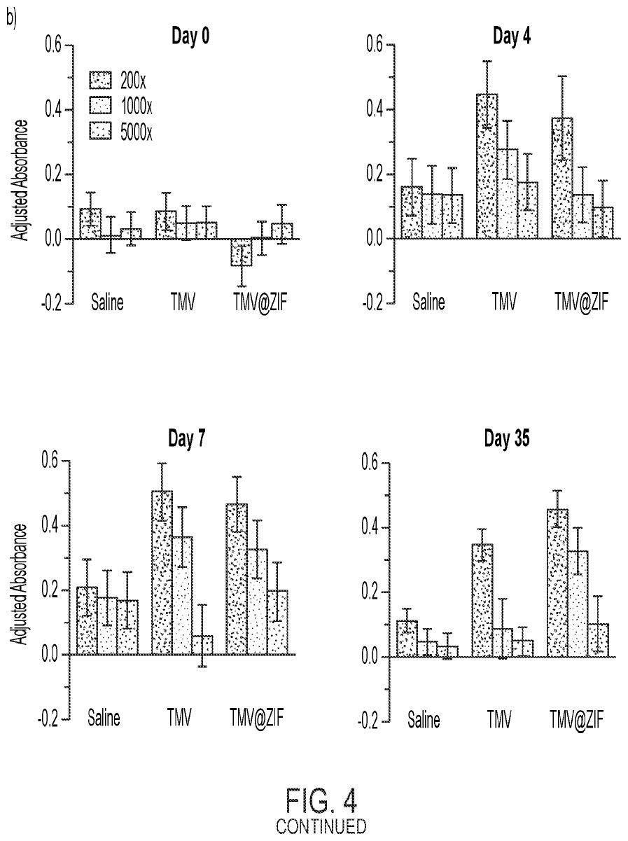

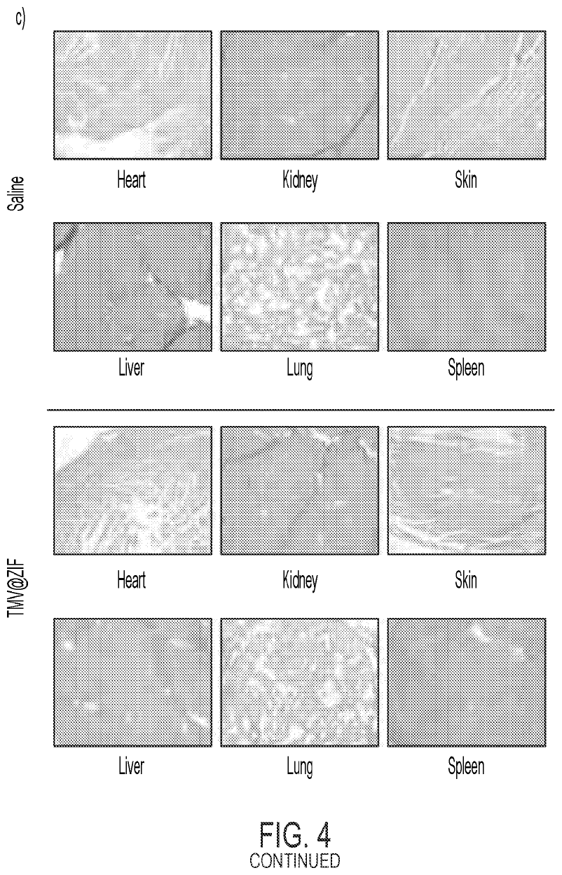

The inventors next turned Their attention to in vivo studies on murine models to determine (i) whether the virus would release from the protective ZIF shell in vivo, (ii) how the anti-TMV IgG production against subcutaneously administered TMV@ZIF compares to native TMV, and (iii) the biocompatibility of the TMV@ZIF composite. In order to determine relative antibody production and optimize serum dilutions, two groups (n =4) of BALB/c test mice were either noninjected or injected subcutaneously with native TMV and blood drawn after 10 days. In the test mice, there was a clear anti-TMV ELISA response in mice injected with native TMV compared to noninjected mice after 10 days, and an optimal serum dilution range of 200× to 5000' was found (Figure S5). To continue the investigation, 12 BALB/c mice were divided into three groups (n =4) and subcutaneously injected on day 0, 2, 4, and 6 with saline, native TMV, or TMV@ZIF. Multiple injections were administered to enhance the antibody production levels in mice. The inventors hypothesized that the TMV@ZIF would protect the encapsulated TMV in the body for as long as native TMV and result in a similar antibody production level. Sub- mandibular blood draws were conducted on day 0, 4, 7, and 35 (FIG. 4A). The ELISA response, which measures the production of mouse antibodies against TMV, shows that the TMV@ZIF elicits an antibody response comparable to naked TMV (FIG. 4B). Antibody production typically depends upon successful uptake by antigen-presenting cells (APCs) for instance macrophages and dendritic cells in the body. This means that the shell is being removed before or during APC uptake. There is literature precedent (Hoop et al., 2018) that ZIF-8 can dissolve in the presence of cell media and it is not unexpected that ZIF-8 would dissociate in the interstitial fluids of the subcutaneous region prior to cellular uptake. The antibody levels that were detected for the composites were comparable to that of naked TMV, confirming that the TMV@ZIF does not need to be exfoliated before administration. 5 days after the last blood withdrawal, H&E-stained images were taken on various organs for each mouse to further evaluate TMV@ZIF biocompatibility. No visual difference could be determined between mice injected with saline and with TMV@ZIF (FIG. 4C). This confirms the biocompatibility of TMV@ZIF, following multiple subcutaneous injections with no apparent toxicity or behavioral changes in the mice. Although some literature has shown in vitro toxicity (Hoop et al., 2018; Zhang et al., 2017), this study has concluded that these results may not translate in vivo.

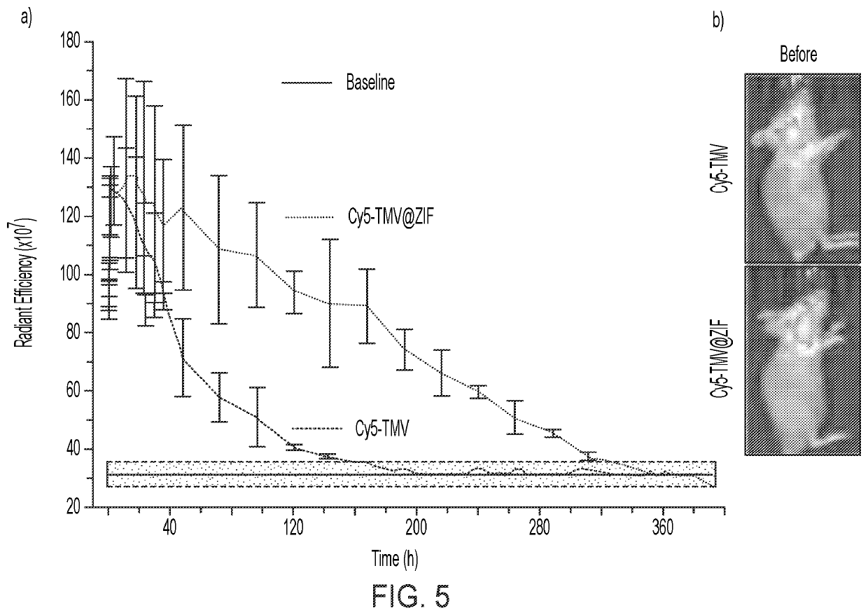

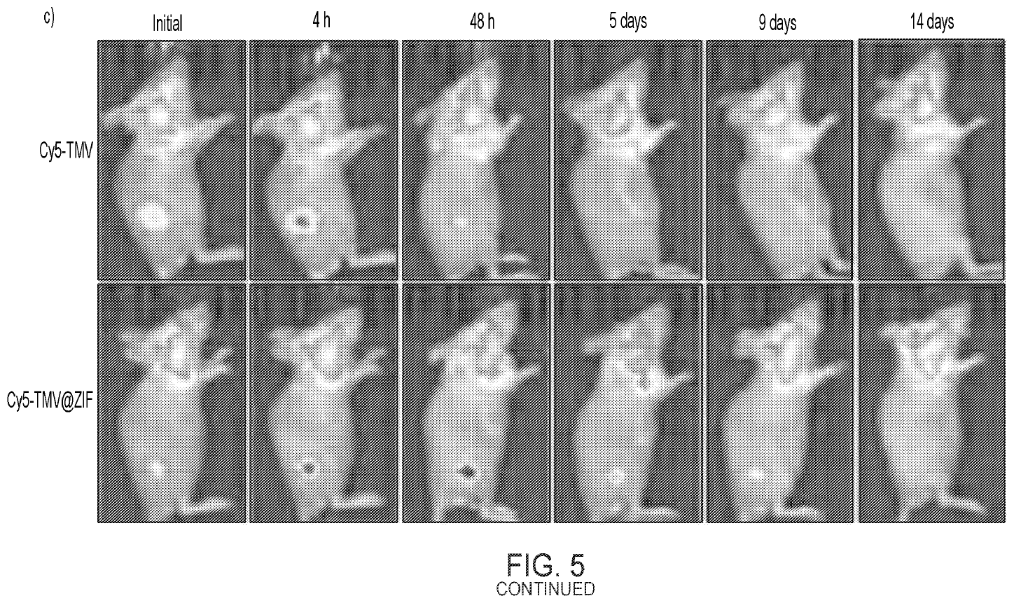

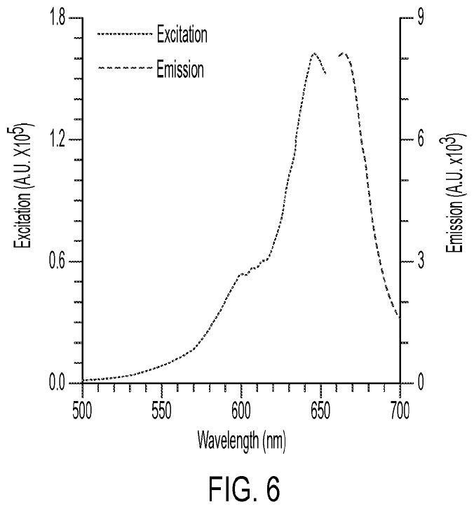

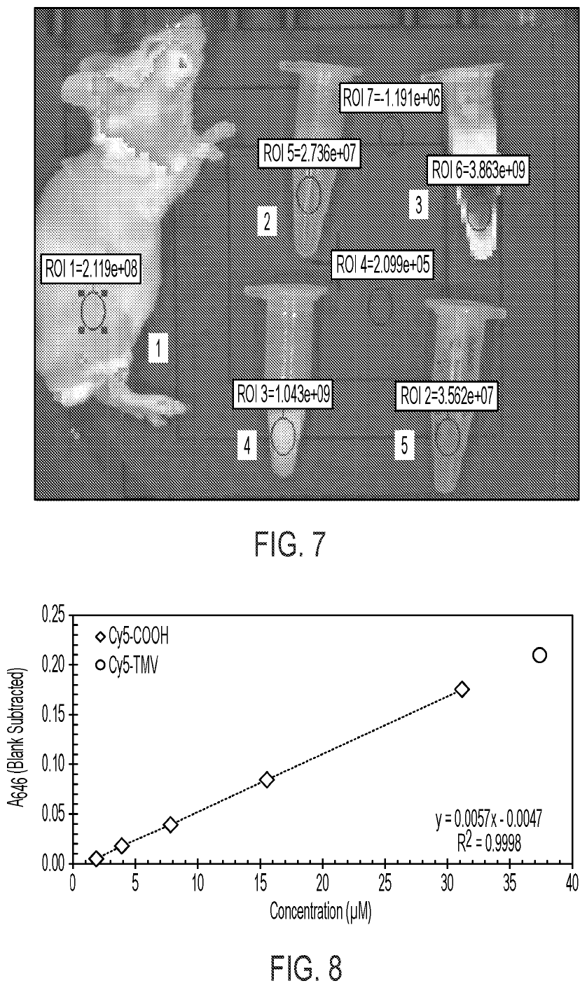

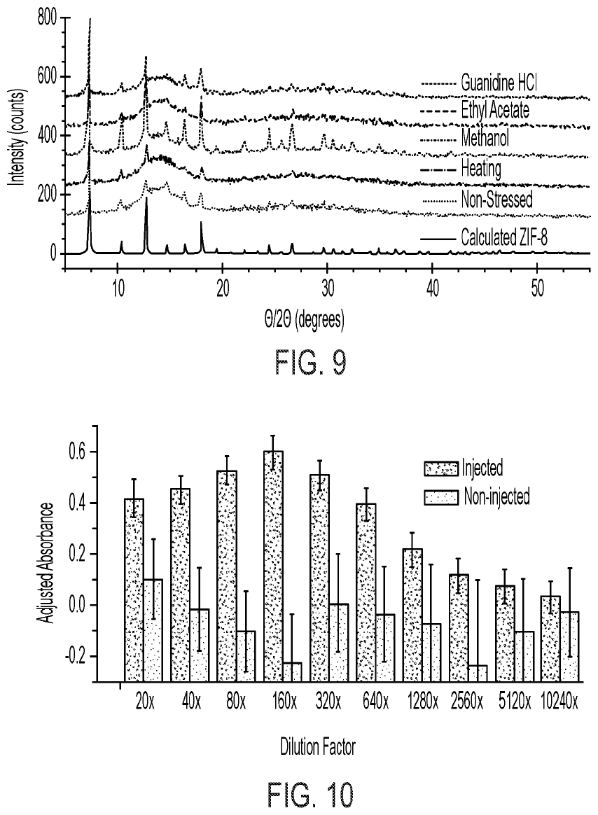

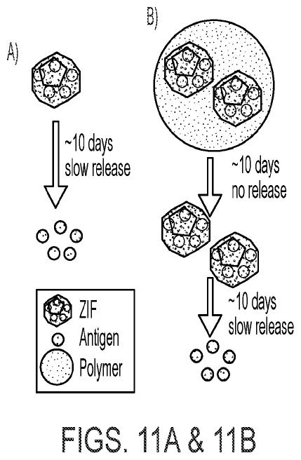

Our method depends upon the slow degradation of ZIF-8 in vivo by physiological salts and macromolecules, suggesting that encapsulation and protein—ZIF composite formation may be an intriguing way to prolong a linear dose of protein-based drugs. This could be especially useful for the administration of insulin and vaccines, which typically require multiple injections over time to achieve a sustained effect (Schade et al., 2017). Histology of the tissue at the subcutaneous site of administration at the conclusion of the study which consisted of four consecutive TMV@ZIF injections did not uncover any residual material, tissue damage, or scarring, which lead us to suspect that the full dose was being absorbed into the animal, as shown in FIG. 4C. To better understand the rate at which TMV@ZIF was taken up by the mouse, the inventors conducted time-dependent in vivo imaging using TMV labeled on its interior with the red-emitting fluorophore Cy5 (Cy5-TMV, λₑₓ=647 nm, λₑₘ=666 nm, FIG. 6). This labeled TMV was encapsulated inside ZIF-8, which caused a quenching of the red fluorescence. This fluorescence of Cy5-TMV was restored in full when the shell was removed, providing a clear indication of shell removal (FIG. 7). For this study, 6 BALB/c mice were divided into two groups (n=3), shaved to remove the hair on their torso and limbs, and injected subcutaneously with either unencapsulated Cy5-TMV or Cy5-TMV@ZIF and imaged over two weeks. The images shown in FIG. 5B show that, prior to injection, the only fluorescence comes from the hairs near the head. As shown in the series of images in FIG. 5C, subcutaneous injection of Cy5-TMV decayed slowly over a period of 120 h. In contrast, the Cy5-TMV@ZIF fluoresced weakly at first, followed by an increase and then gradual decay. After 288 h, the fluorescence at the injection site for the encapsulated material returned to the baseline.

The results of this study demonstrate that ZIF-8 coatings not only provide protection against denaturing solvents and heat, but the nucleation and growth of the crystalline lattice does not alter the secondary or tertiary structure of protein and protein ensembles. Further, the shell does not significantly damage the viral RNA. The ZIF-8 shells are simple to synthesize on proteins, their composites are formed in a few seconds, and are ready to use within hours. It is clear from histology data that prolonged exposure to ZIF-8 does not alter the tissue morphology at either the injection site or distal organs. Qualitatively, the inventors saw no behavior changes in mice following administration, nor did any mice become ill or die as a result of prolonged exposure to TMV@ZIF composites. On the other hand, in vivo data clearly suggest that the administration of equal quantities of immunogenic protein yielded identical antibody responses, showing that the release of the protein from the ZIF shell occurs to completion. This was further corroborated with time-dependent in vivo imaging studies, which showed a time-delayed release of the injection over the course of 14 days a property of ZIF-8 the inventors aim to exploit in subsequent studies. Taken together, these data strongly suggest that ZIF-8-based shells may provide a method to concurrently protect and deliver proteinaceous drugs safely.

### C. Materials

Materials. Acetic acid, acetic anhydride, acetone, bovine serum albumin, 6-bromohexanoic acid, 1- butanol, chloroform, o-dichlorobenzene, diethanolamine, egg albumin, ethyl acetate, 1-ethyl-3-(3-dimethylaminopropyl)carbodiimide (EDC), ethylenediamine, ethylenediaminetetraacetic acid (EDTA), guanidinium chloride, hydrazinobenzene sulfonic acid hydrates, hydrochloric acid, hydroxybenzotriazole (HOBt), 4-(2-hydroxyethyl)piperazine-1-ethanesulfonic acid (HEPES), iodomethane, magnesium chloride, malonaldehyde bis(phenylimine) monohydrochloride, β-mercatoethanol, methanol, 3-methyl-2-butanone, 2-methylimidazole, paraformaldehyde, p-nitrophenylphosphate, potassium hydroxide, potassium phosphate dibasic, potassium phosphate monobasic, poly(ethylene glycol) 8000, poly(vinylpyrrolidone) 40k (PVP 40k), 2-propanol, pyridine, sodium azide, sodium bicarbonate, sodium carbonate, sodium chloride, sodium hydroxide, sodium phosphate dibasic, sodium phosphate monobasic, sodium sulfite, sucrose, Triton X-100, Tween-20, and zinc acetate dihydrate were purchased from Sigma-Aldrich (St. Louis, Mo., USA), Thermo Fisher Scientific (Waltham, Mass., USA), Chem-Impex Int'l (Wood Dale, Ill., USA), or VWR (Radnor, Pa., USA), and used without further modification. Lowry assay was performed using a Pierce Modified Lowry assay kit (Thermo Fisher Scientific). ELISA was performed using a TMV ELISA kit (Agdia Inc. Elkhart, Ind., USA). Rabbit antiTMV IgG and Rabbit antiTMV-alkaline phosphatase IgG were provided with the ELISA kit. Goat antimouse-alkaline phosphatase IgG was purchased from Sigma-Aldrich. Cy5-COOH was synthesized according to a literature protocol (Dharmarwardana et al., 2018). Ultrapure water was obtained from an ELGA PURELAB flex 2 system with resistivity measured to at least 18.2 MΩ-cm.

ELISA Buffers. ELISA buffers were prepared according to documentation provided with the TMV ELISA kit. Coating buffer: pH 9.6 Sodium carbonate/bicarbonate with sodium azide Wash buffer: 0.1 M pH 7.4 PBS with 0.2% Tween-20 Sample Extraction buffer: Wash buffer with PVP 40k, sodium sulfite, chicken egg albumin, and sodium azide Conjugate buffer: Wash buffer with bovine serum albumin, PVP 40k, and sodium azide Substrate buffer: 1 M pH 9.8 diethanolamine with magnesium chloride and sodium azide

UV-Vis. UV-Vis spectra were taken using a UV-1601PC UV-Vis-NIR Spectrophotometer (Shimadzu, Kyoto, Japan), Tecan Spark 20M plate reader (Tecan, Mannedorf, Switzerland), or Biotek Synergy H4 hybrid reader (Biotek, Winooski, Vt., USA). NanoDrop UV-Vis measurements were performed on a Thermo Scientific NanoDrop 2000 Spectrophotometer.

Fluorescence. Fluorescence measurements were taken using a Tecan Spark 20M plate reader.

Scanning Electron Microscopy. SEM was performed on a ZEISS Supra 40 Scanning Electron Microscope (Zeiss, Oberkochen, Germany) with an accelerating voltage of 2.5 kV and a working distance of 6.7 to 15.3 mm Samples were sputtered with a 37 Å layer of gold.

Transmission Electron Microscopy. Transmission electron micrographs were taken on a JEOL JEM-1400+ (JEOL, Tokyo, Japan) at 120 kV with a Gatan 4k×4k CCD camera. 5 μL of the ˜0.1 mg/mL desalted sample was placed on a 300 mesh Formvar/carbon-coated copper grid (Electron Microscopy Sciences, Hatfield, Pa., USA), allowed to stand for 30 seconds, and wicked off with Whatman #1 filter paper. 5 μL of 2% uranyl acetate (SPI Supplies, West Chester, Pa., USA) was placed on the grid, allowed to stand for 30 seconds, wicked off as before, and the grid allowed to dry completely in air.

In vivo Fluorescence Imager. Fluorescent animal imaging was taken with IVIS Lumina III (PerkinElmer, Waltham, Mass., USA) at an excitation of 620 nm and emission at 670 nm with a 0.5 s exposure.

Powder X-Ray Diffraction. PXRD was taken on a Rigaku SmartLab diffractometer with CuKα (1.54060 Å) at 40 kV and 30 mA. The scans were performed for 2θ from 5° to 55° with a step size of 0.01°.

### D. Methods

Preparation of TMV@ZIF. A 0.111 mg of TMV was briefly mixed with 3 mL of 400 mM 2-methylimidazole and to this solution was rapidly added 1.5 mL of 20 mM zinc acetate and the solution was swirled for 20 s. Flocculates appeared within a few seconds. The solution was left on the bench at R.T. for 16-18 h, then centrifuged at 4300 g for 20 mM and the supernatant was discarded. The pellet was washed twice with ultrapure water and used as is.

Propagation and Isolation of TMV. TMV particles were isolated from Nicotiana benthamiana plants from a previously published method (Li et al., 2016). The tobacco plants were grown, infected, and collected after 10 d of infection and stored at −80° C. until needed. Approximately 100 g of leaves were blended in pulses with 1000 mL of ice-cold extraction buffer (0.1 M pH 7.4 potassium phosphate (KP) buffer, 0.2% (v/v) β-mercaptoethanol) followed by being pulverized with a mortar and pestle. The mixture was filtered through cheesecloth to remove the plant solids, and the filtrate centrifuged at 11,000×g for 20 min at 4° C. The supernatant was filtered through cheesecloth again, and an equal volume of 1:1 chloroform/1-butanol mixture was added and stirred on ice for 30 min. The mixture was centrifuged at 4500×g for 10 min. The aqueous phase was collected, followed by the addition of NaCl to a final concentration of 0.2 M, 8% (w/w) PEG 8000, and 1% (w/w) Triton X-100 surfactant. The mixture was stirred on ice for 30 min and stored at 4° C. for 1 h. The solution was centrifuged at 22,000×g for 15 min at 4° C. The supernatant was discarded, and the pellet resuspended in 0.1 M pH 7.4 potassium phosphate (KP) buffer at 4° C. overnight.

The supernatant was carefully layered on a 40% (w/v) sucrose gradient in 0.01 M KP buffer (with at least one freeze-thaw cycle) in ultraclear tubes and centrifuged in a swing bucket rotor for 2 h at 96,000×g. The light-scattering region was collected and centrifuged at 360,562×g for 1.5 h. The supernatant was discarded, and the pellet resuspended in 0.01 M pH 7.4 KP buffer overnight. The solution was portioned equally into microcentrifuge tubes and centrifuged at 15,513×g for 15 min. The supernatant was collected as the final TMV solution. UV-Vis measurements were taken with NanoDrop at 260 nm (RNA) and 280 nm (protein). A ratio of A260/A280 around 1.23 indicates intact TMV. Using the Beer-Lambert Law with ε=3 as reported (Li et al., 2016), the solution concentration was determined.

Synthesis. Cy5-COOH was synthesized according to literature protocol (Park et al., 2012), and the procedure is reproduced below from Dharmarwardana et al., 2018.

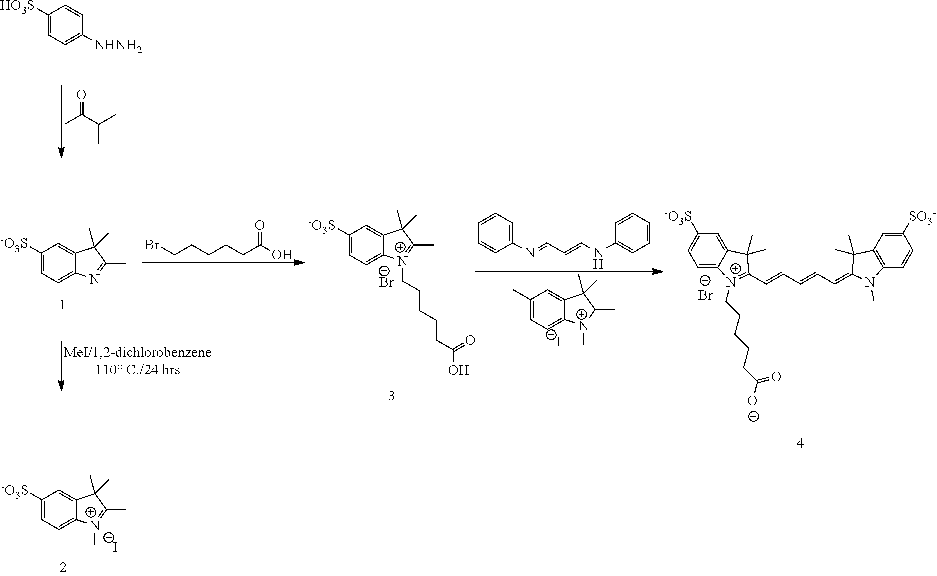

Synthesis of 3H-Indole-2,3,3-trimethyl-5-sulfonic Acid, Potassium Salt (1). Hydrazinobenzene sulfonic acid hydrates (1.50 g, 7.60 mmol) and 3-methyl-2-butanone (2.52 mL, 23.4 mmol) were dissolved in acetic acid (4.5 mL). The mixture was heated to reflux at 110° C. for 3 h and acetic acid was removed. A solution of crude sulfonic acid in methanol (10 mL) was added dropwise to a stirred solution of potassium hydroxide (0.500 g) in 2-propanol (10 mL). The resulting mixture was stirred at 25° C. for 24 h and filtered through a paper filter. The residue was dried under reduced pressure to provide the crude compound (1.02 g, 4.49 mmol, 59.1% yield) (1). ¹H-NMR (600 MHz, D20) δ ppm 1.237; (s, 6H), 2.259; (s, 3H), 7.494; (d, J=8.02, 1H), 7.764; (d, J=8.09, 1H), 7.792; (s, 1H).

Synthesis of 3H-Indolium, 1-Methyl-2,3,3-trimethyl-5-sulfonate (2). A slurry of crude 1 (0.900 g, 3.24 mmol) in iodomethane (3.5 mL, 0.20 mol) under N2 was heated to reflux for 24 h and cooled down to 25° C. The liquid phase was decanted, and the residue was washed with acetone (3×50 mL), filtered with a paper filter, and dried under reduced pressure at 40° C. to afford the crude compound (0.440 g, 1.25 mmol, 38.7% yield) (2). 1H-NMR (600 MHz, D20) δ ppm 1.522; (s, 6H), 2.153; (s, 3H), 3.971; (s, 3H), 7.777; (d, J=8.25, 1H), 7.963; (d, J=8.80, 1H), 8.026; (s, 1H).

Synthesis of 3H-Indolium, 1-(5-Carboxypentyl)-2,3,3-trimethyl-5-sulfonate (3). 3H-Indole-2,3,3-trimethyl-5-sulfonic Acid, Potassium Salt (1.02 g, 3.70 mmol) and 6-bromohexanoic acid (0.827 g 4.33 mmol) were suspended in o-dichlorobenzene (5 mL). The suspension was stirred at 110° C. for 19 h, then allowed to cool to room temperature, and the supernatant was removed to afford 3H-Indolium, 1-(5-Carboxypentyl)-2,3,3-trimethyl-5-sulfonate (1.10 g, 2.73 mmol, 73.80% yield) (3). 1H-NMR (600 MHz, D20) δ ppm 1.378; (m, 2H), 1.586; (s, 6H), 1.601; (m, 4H), 1.904; (t, J=7.65, 2H), 2.301; (t, J=7.14, 2H), 4.433; (t, J=7.41, 2H), 7.818; (d, J=8.58, 1H), 7.945; (d, J=8.64, 1H), 7.952; (s, 1H).

Synthesis of 3H-Indolium, 2-[5-[1-(5-Carboxypentyl)-1,3-dihydro-3,3-dimethyl-5-sulfo-2H-indol-ylidene]-1,3-pentadien-1-yl]-1-methyl-3,3- dimethyl-5-sulfonate (4). A suspension of 1-methyl-2,3,3-trimethyl-3H-indol-1-ium-5-sulfonate (2) (0.253 g, 1.00 mmol) and malonaldehyde bis(phenylimine) monohydrochloride (0.235 g, 1.10 mmol) in acetic acid (5 mL) and acetic anhydride (5 mL) was refluxed at 110° C. for 4 h. Solvent was removed under reduced pressure and the resultant solid was dissolved in pyridine (10 mL) under N2. The mixture was then treated with compound (3) (0.353 g, 1.00 mmol) at 25° C. Reaction mixture was stirred at 60° C. for 4 h, cooled down to 25° C., and agitated a heterogeneous mixture by addition of ethyl acetate (10 mL). Resulting mixture was filtered through a paper filter, and the residue was dried under reduced pressure and purified with reverse flash chromatography to yield (4) as a dark blue solid (0.150 g, 0.234 mmol, 23.4% yield). 1H-NMR (600 MHz, D20) δ ppm 1.372; (m, 2H), 1.539; (m, 14H), 1.716; (p, J=4.90, 4H), 2.294; (t, J=7.50, 2H), 3.518; (s, 3H), 3.964; (m, 2H), 6.0474; (t, J=14.03, 2H), 6.376; (t, J=12.19, 1H), 7.259; (t, J=6.78, 2H), 7.741-7.779; (m, 3H), 7.871; (t, J=12.72, 2H).

Preparation of TMV@ZIF composites. TMV@ZIF was prepared according to literature protocol (Li et al., 2016). 0.111 mg of native TMV was added to a 20 mL scintillation vial, followed by 3 mL of 400 mM 2-methylimidazole in 3 1 mL aliquots. 3×500 μL aliquots of 20 mM zinc acetate dihydrate were rapidly added to the virus-ligand solution, and the vial capped and swirled for 20 sec. Flocculates appeared within the first few seconds of zinc addition. The solution was left to incubate on the benchtop at R.T. for 16 to 18 h. The ripened solution was then transferred to a 15 mL Falcon tube and centrifuged at 4300×g for 20 mM at 4° C. The supernatant was discarded, and the pellet washed with ultrapure water twice. The final TMV@ZIF powder was then ether used as-is or dried in air.

Bioconjugation. Interior-modified Cy5-TMV was prepared according to a previously reported method (Dharmarwardana et al., 2018). The interior surface of TMV was modified with ethylenediamine (EA) using an EDC coupling reaction. 200 μL of a TMV solution (10 mg/mL) was diluted to 2 mg/mL with 574 μL of 0.1 M pH 7.4 HEPES buffer at R.T.

followed by the addition of 130 μL of 0.1 M EA, 3 mg of HOBt, and 96 μL of 0.1 M EDC. The reaction mixture was incubated at R.T. for 24 h, purified with a PD MidiTrap G-25 column, and the solution was washed three times with 0.1 M KP buffer and concentrated to 10 mg/mL with a 10K MWCO Pierce™ Protein Concentrator to yield inEA-TMV. Cy5-COOH was then attached by the EDC reaction. 200 μL of the inEA-TMV solution (10 mg/mL) was diluted to 2 mg/mL with 574 μL of 0.1 M pH 7.4 HEPES buffer at R.T. followed by the addition of 130 μL of 0.1 M Cy5-COOH, 3 mg of HOBt, and 96 μL of 0.1 M EDC. The reaction mixture was incubated at R.T. for 24 h, purified with a PD MidiTrap G-25 column, and the solution was washed three times with 0.1 M KP buffer and concentrated to 10 mg/mL with a 10K MWCO Pierce™ Protein Concentrator to yield Cy5-TMV.

Preparation of Cy5-TMV@ZIF. Cy5-TMV@ZIF was prepared using the same protocol as TMV@ZIF, except using Cy5-TMV instead of native TMV. The Cy5-TMV concentration was determined by NanoDrop to be 12.59 mg/mL and the apparent Cy5 concentration by UV-Vis was 37.44 μM. Calculating the number of moles of Cy5 and TMV present in the Cy5-TMV (TMV M.W.=4.08×107 g/mol, Cy5 M.W.=746.97 g/mol), the average number of Cy5 molecules per TMV rod was determined to be 12.13. Fluorescence and UV-Vis absorption characterizations are shown in FIG. 8.

EDTA Exfoliation. Exfoliation buffer was prepared by adding EDTA to 0.1 M in a 0.1 M potassium hydroxide solution. Solid potassium hydroxide pellets were added until the

EDTA was fully dissolved, then the pH adjusted to 7.0 with HC1. TMV@ZIF composites were exfoliated by reducing the solvent level to a minimum or drying out, then adding 1 to 2 mL of EDTA Exfoliation buffer and left on a rotisserie at 37° C. Wet samples became water-clear within the first few minutes. Resuspended dried samples became cloudy and required a longer time to clear up, up to overnight. Samples were then buffer exchanged with a 10K MWCO Pierce™ Protein Concentrator.

ELISA:

Stressed TMV. Stressed TMV@ZIF samples were exfoliated, then both exfoliated stressed TMV@ZIF and stressed naked TMV samples were desalted with a 10K MWCO Pierce™ Protein Concentrator and resuspended in 0.1 M pH 7.4 sodium phosphate buffer. Protein concentrations were then determined by Lowry assay before being diluted to 5×10-4 mg/mL for ELISA.

Rabbit anti-TMV IgG in coating buffer was added 100 μL per well to a 96-well plate and incubated at R.T. for 4 h or overnight at 4° C. The plate was emptied and washed 3× with wash buffer. Samples and standards—concentrations determined by Lowry assay—were diluted to 5×10-4 mg/mL with sample extraction buffer, added 100 μL per well with additional wells filled with 100 μL per well with just sample extraction buffer as the buffer blank, and incubated for 2 h at R.T. or overnight at 4° C. The plate was emptied and washed 8× with wash buffer. Alkaline phosphatase-conjugated rabbit anti-TMV IgG in conjugate buffer was added 100 μL per well and incubated for 2 hours at R.T. The plate was emptied and washed 8× with wash buffer. 1 mg/mL p-nitrophenylphosphate in substrate buffer was added 100 μL per well and the plate developed for 45 min at R.T. The plate was read at 405 nm, 420 nm, and 450 nm, and the absorbance values of buffer blank wells averaged and subtracted from the entire plate. Experiments were performed in 4 replicates and the values were normalized between that of a separate internal control of naked non-stressed TMV (100%) and that of the buffer blank (0%). The internal control ensures data comparability between separate ELISA experiments. Percent difference was calculated according to the equation:

$$\frac{\vert {V}_{1} - {V}_{2} \vert}{( \frac{{V}_{1} + {V}_{2}}{2} )}$$

where V1 and V2 are values expressed in percentages. The percent differences are listed in Table 1.

Plant Infection. Nicotiana benthamiana plants were divided into 4 groups (n=6 plants) and inoculated with 0.01 M pH 7.4 KP buffer as a negative control, TMV@ZIF in ultrapure water, TMV@ZIF exfoliated with EDTA and buffer exchanged into 0.01 M pH 7.4 KP buffer, and native TMV in 0.01 M pH 7.4 KP buffer as a positive control. Solutions were prepared such that 50 μL of solution delivered 5 μg of TMV, and 50 μL per leaf was used. To ensure no cross contamination each group was placed in different trays and watered and handled separately. After 10 d, the plants were collected into separate bags and stored at −80° C. until needed. Frozen leaves were coarsely ground and approximately 1 g of recovered plant matter per group was macerated using a mortar and pestle in 10 mL of sample extraction buffer per 1 g of leaves. The plant pulp was allowed to extract overnight at 4° C., then centrifuged to remove large plant matter, and the supernatant collected as samples for ELISA.

Rabbit anti-TMV IgG in coating buffer was added 100 μL per well to a 96-well plate and incubated at R.T. for 4 h or overnight at 4° C. The plate was emptied and washed 3× with wash buffer. The collected plant extraction solutions were added 100 μL per well in 1×, 10×, and 50× dilutions, and incubated for 2 h at R.T. or overnight at 4° C. The plate was emptied and washed 8× with wash buffer. Alkaline phosphatase-conjugated rabbit anti-TMV IgG in conjugate buffer was added 100 μL per well and incubated for 2 h at R.T. The plate was emptied and washed 8× with wash buffer. 1 mg/mL p-nitrophenylphosphate in substrate buffer was added 100 μL per well and the plate developed for 45 min at R.T. The plate was read at 405 nm, 420 nm, and 450 nm, and the absorbance values of buffer blank wells averaged and subtracted from the entire plate. Experiments were performed in 4 replicates, a best-fit line was fit to the blank-subtracted averaged standard values, sample values were calculated from the equation, dilutions were back-calculated and averaged, and values reported as the average±standard deviation of the apparent sample concentrations in μg/mL.

Mouse serum. Rabbit anti-TMV IgG in coating buffer was added 100 μL per well to a 96-well plate and incubated at R.T. for 4 h or overnight at 4° C. The plate was emptied and washed 3× with wash buffer. Native TMV standards (concentrations determined by Lowry assay) were diluted to 0.0005 mg/mL with sample extraction buffer, added 100 μL per well, and incubated for 2 h at R.T. or overnight at 4° C. The plate was emptied and washed 8× with wash buffer. Mouse serum was diluted 200×, 1000×, and 5000× in sample extraction buffer, 100 μL per well was added, and incubated at R.T. for 2 h. The plate was emptied and washed 8× with wash buffer. Alkaline phosphatase-conjugated goat anti-mouse IgG in conjugate buffer was added 100 μL per well and incubated for 2 h at R.T. The plate was emptied and washed 8× with wash buffer. 1 mg/mL p-nitrophenylphosphate in substrate buffer was added 100 μL per well and the plate developed for 45 min at R.T. The plate was read at 405 nm, 420 nm, and 450 nm, and the absorbance values of the buffer blank wells averaged and subtracted from the entire plate. The blank-subtracted values of each mouse group were reported as the average±standard deviation for each dilution.

Test Mice. 8 BALB/c mice were divided into two groups (n=4) and either left uninjected or injected subcutaneously with 100 μg of native TMV in saline. Blood was drawn submandibularly after 10 d, centrifuged to remove erythrocytes, and relative antiTMV IgG levels determined by ELISA as per the above procedure. Serum was serially diluted by factorsof 2 from 20× to 10240× in order to determine relative ELISA responses and optimal serum dilution levels for subsequent ELISAs.

Mouse Time Study. All mice studies were approved by the Institutional Animal Care and Use Committee at the University of Texas at Dallas (Protocol # 17-05). 12 BALB/c mice were divided into three groups (n=4) and injected with saline, native TMV in saline, or TMV@ZIF suspended in saline. TMV solutions were prepared such that 200 μL delivered 10 μg of TMV. Doses of 200 μL of saline, TMV, or TMV@ZIF were administered subcutaneously on day 0, 2, 4, and 6 and blood was withdrawn submandibularly on day 0, 4, 7, and 35. The blood was centrifuged to remove erythrocytes, and the antiTMV IgG content of the resultant serum was determined by ELISA as described above. At the end of the study, the mice were sacrificed for histological analysis on the spleen, liver, kidney, lung, heart, and the skin at the administration site. The mice were sacrificed by carbon dioxide asphyxiation, the organs harvested, and fixed in 4% formaldehyde overnight. The fixed organs were moved to a 70% ethanol solution and processed with an ASP300 S tissue processor (Leica Biosystems, Buffalo Grove, Ill.) for dehydration into paraffin. The organs were then embedded into paraffin wax using a HistoCore Arcadia C and H paraffin embedding station (Leica Biosystems, Buffalo Grove, Ill.). Each organ was sliced into 4 μm sheets using a RM2235 manual microtome (Leica Biosystems, Buffalo Grove, Ill.) and imaged with a DMi1 optical microscope (Leica Biosystems, Buffalo Grove, Ill.) at 40× magnification.

Mouse Imaging. Ten BALB/c mice were fed a non-fluorescent diet and shaved once the mice showed no abdominal autofluorescence. The mice were anaesthetized with isoflurane and injected with 200 μL of saline (n=4), Cy5-TMV (n=3), or Cy5-TMV@ZIF (n=3). The TMV-containing solutions were prepared such that 200 μL delivered 10 μg of TMV. A series of time points were taken at 1, 5, 10, and 30 min, with additional time points at 1, 2, 4, 8, 12, 18, 24, 30, 36, 48, and every 24 h thereafter until the fluorescence decayed back to the average level of the saline injected mice.

TMV@ZIF Stressing. Three batches of TMV@ZIF were combined and either left as is (non-stressed), soaked in 1 mL of methanol, ethyl acetate, or 6 M guanidinium chloride overnight, or heated to 100° C. in a water bath for 20 min Naked TMV samples were stressed in the same manner, with 0.333 mg of TMV soaked in 1 mL solvent overnight, or heated to 100° C. for 20 min. Encapsulated samples were collected via centrifugation at 4300 g for 20 min, rinsed with ultrapure water, and exfoliated in EDTA overnight. Exfoliated and naked samples were buffer exchanged into 0.1 M pH 7.4 sodium phosphate buffer in a centrifugal filter for concentration determination by the Lowry assay and then diluted to 5×10-4 mg/mL for ELISA measurements.

Plant Inoculation. N. benthamiana plants were divided into four groups (n=6) and inoculated with either 0.1 M pH 7.4 potassium phosphate buffer (negative control), naked TMV in potassium phosphate buffer (positive control), TMV@ZIF in ultrapure water, or exfoliated TMV@ZIF in potassium phosphate buffer. Solutions were prepared such that 50 μL of solution delivered 5 μg of TMV, and 50 μL per leaf was used. Plants were collected after 10 d, macerated in buffer, soaked overnight, and centrifuged to remove large plant matter. The apparent TMV concentrations of the supernatants were determined by ELISA.

In vivo Antibody Response. BALB/c mice were divided into three groups (n=4), and subcutaneously injected with 200 μL of saline, naked TMV in saline, or TMV@ZIF in saline. TMV solutions were prepared such that 200 μL delivered 10 μg of TMV. Injections were administered on days 0, 2, 4, and 6, and submandibular blood draws were taken on days 0, 4, 7, and 35. Blood was centrifuged to remove erythrocytes and the resulting serum anti-TMV IgG levels were determined by ELISA. At the conclusion of the study, the mice were sacrificed, and histological analysis was performed.

In vivo Fluorescence Imaging. BALB/c mice were fed a nonfluorescent diet and shaved to remove autofluorescence, divided into three groups and injected with 200 μL of saline (n=4), Cy5-TMV (n=3), or Cy5-TMV@ZIF (n=3). The TMV-containing solutions were prepared such that 200 μL delivered 10 μg of TMV. In vivo fluorescence images were taken at a series of timepoints until the fluorescence levels of the Cy5-injected mice returned to the baseline levels of the saline-injected mice.

### Example 2—Encapsulation of ZIF-Antigens in a Slow-Releasing Polymer for Extended Release

Controlled release of antigens is associated with long-term T-cell memory and possibly the elimination of booster shoots Eliminating booster shots would reduce the number of injections needed to create long-lasting immunity, reduce patient costs, and increase patient compliance. Based on their preliminary results with VLP@ZIF, the inventors anticipate that antigen@ZIF, when placed into the body, will release proteins for approximately 10 days. The objective of this aim is to create a “delayed release” formulation of that will not release anything (or release proteins very slowly) for the first 10 days and, after all the uncoated antigen@ZIF is exhausted, begin releasing antigen (FIG. 11). The inventors' working hypothesis is that they can imbed the antigen@ZIF into a polymer, which will prevent release of any antigens for a programmable number of days. Co-administration of the antigen@ZIF and antigen@zif@polymer will provide approximately 20 days of sustained antigen release. After 11 weeks, the inventors expect the same IgG titers following a single administration of a commixture of antigen@ZIF@polymer and antigen@ZIF as compared to multiple injections of the unencapsulated antigen. Conceptually, the antigen will be first embedded in the ZIF, which would protect it from processing. The inventors will use different ester-based polymers to encase the antigen@ZIF formulations. Each polymer was selected because its in vivo dissolution has been comprehensively studied. For instance, as polylactic acid (PLA), poly(lactic-co-glycolic acid) (PLGA) and polycaprolactone (PCL) are known for their burst release kinetics, which is actually what the inventors want—after the degradation of the polymer, the antigen@ZIF should come out and begin its gradual dissolution and release of antigen.

Encapsulation of antigens in plastic. In addition to antigen stability outside of cold storage, the control over delivery time and rate is a critical parameter toward vaccine efficacy. While ZIF coating has been proven to be an effective method for stabilizing protein structure, its rate of degradation can difficult to regulate. To address this issue, the inventors propose to encapsulate the proposed antigen@ZIF formulations in polymeric nanoparticles. Thermoplastic polyester microparticles are easy to prepare, degradable, and many aliphatic polyesters such PLA, PLGA, and polycaprolactone PCL are biocompatible. These polymers have been previously used in controlled protein delivery applications. The inventors hypothesize that in addition to the temperature stability provided by the ZIF coating, the inventors can also provide controlled delivery through encapsulation in a degradable polymer coating.

Experimental Approach:

Polymer Particle Preparation and Characterization. A variety of techniques for the preparation of loaded polyester microparticles with size control have been described79,81,82 some of which have incorporated porous metal coordination compounds similar ZIFs.83 The polymers (PLGA and PCL) will initially be obtained from commercial sources as polyesters with ˜10 kDa-˜80 kDa. A typical experiment would be to use a oil/water emulsion method to produce a distribution of particle sizes (0.5-20 μm). Briefly: the starting polymer is dissolved in a low-boiling organic solvent (such as dichloromethane) along with ˜500 nm antigen(Cy5)@ZIF nanoparticles. To this solution, water is added and will form a second, non-miscible layer on top. This immiscible solution is stirred very quickly with a motorized stirrer at ambient temperatures such that the immiscible organic solvent is forced into the aqueous layer as nanodroplets containing polymer and antigen(Cy5)@ZIF-8. Faster stir rates result in smaller particles in the emulsion while slower stir rates produce larger particles. Over the course of this experiment, the organic solvent will slowly evaporate from the droplets (while stirring in the aqueous solution) leaving the antigen@ZIF-8 stuck in polymeric nanoparticles. The final antigen(Cy5)@ZIF@polymer can be collected by centrifugation. These particles will have some polydispersity in size and which can be separated by gradient centrifugation. Particle sizes will be characterized using scanning electron microscopy (SEM). PXRD will be used to confirm ZIF-8 has remained in the polymeric matrix.

Cargo Release Rates. A solution of the antigen(Cy5)@ZIF@polymer formulations of similar diameter will be isolated inside a 0.22 μM membrane at the bottom of a quartz cuvette below the beam path of a fluorimeter. The membrane porosity is small enough to prevent microparticle release but will allow antigen(cy5) diffusion into the beam path as the plastic and ZIF-8 dissolve. Fetal Bovine serum (FBS) at 37° C. or simulated body fluid (SBF) will be added and fluorescence will be monitored over the course of a month. As the polymers degrade, the drugs will enter solution and their concentrations can be elucidated from a calibration curve. The objective is to create a formulation that delays release by approximately the same amount of time it takes for antigen@ZIF to fully dissolve. The same assay will be conducted for antigen@ZIF (not encapsulated in polymer) to generate comparable results.

Check proteins. To determine yield of encapsulation the supernatant from the emulsion reaction, the pellet from the centrifugation will be treated with EDTA for 12 hours.

This is sufficient time to exfoliate the ZIF to remove the antigen but should not release any antigens that are hidden inside the polymer. Total protein concentration will then be assayed. To confirm proteins inside the Antigen@ZIF@polymer remain folded, the polymer composite will be soaked in pH 8 water until the polymer shell dissolves to isolate the freed antigen@ZIF. This will then be subjected to the battery of tests described in aim 1 to confirm structure. Antigen@ZIF@polymer will be subjected to the same experimental battery as antigen@ZIF described in Aim 2 to obtain in vivo and in vitro data.

Controls. The inventors will measure antigen@ZIF release rates in FBS or SBF. They will also compare hydrolysis rates of the polyester microparticles with and without embedded antigen@ZIF. Dissolution of polyester particles without antigen(Cy5)@ZIF will be prepared in a solution of FITC. The polyester will thus slowly release FITC over time. Since the crystalline viral nanoparticles will create inhomogeneous structure in the microparticles, it is likely that the rates of release will be non-linear with increasing antigen@ZIF concentration. It is anticipated that it will be possible to make microparticles for subcutaneous implant that will show no or very little release initially and for a pre-set number of days followed by sustained release. The Gassensmith and Smaldone groups have collaborated previously on slow release trans-dermal microneedle plastics derived from polyesters and have experience in designing fluorescence-based assays to monitor their release in (i) conditions that mimic interstitial fluid and (ii) within actual skin. Tuning the synthetic conditions may be necessary to achieve the desired release rates.

Alternative approaches. The inventors anticipate antigen@ZIF loading into the microspheres to be high. ZIF-8 is hydrophobic and preferentially partitions into organic layers. Consequently, the inventors do not anticipate low yielding formation or problems with encapsulation as microemulsion techniques are well developed.23,78,87-90 In the inventors' hands, ZIF-8 nanoparticles below 1 um in diameter are easily suspended in aqueous solution and should actively partition into the organic phase of the emulsion. An alternative approach, however, would be to create microparticles using electrospray methods, of which there are many as this is likewise a developed area of synthesis. With regards to the cargo release rates, there could be two problems—release that is too slow, or too fast. Beyond making the polymer shells larger, to reduce the speed of release, PLA particles could be used. PLA is a harder polyester, with greater resistance to hydrolysis under biological conditions while retaining its biocompatibility. Release that is too slow could be remedied by using either low molecular weight polyesters, or by modifying the preparation method to include porosity or increased surface area which will result in faster particle hydrolysis. If the degradation rates of the microparticles need further tuning, other additives to the polyester matrix, such as chitosan can be used to increase degradation without sacrificing biocompatibility.

Testing encapsulated implants in animal models. In vivo testing is necessary to validate the inventors' observations in vitro. To do that, the inventors will test antigen@ZIF@polymer and show that mouse models produce no antibody response for several days while the polymeric shell is dissolving. Once the antigen@ZIF is free, it will slowly dissolve and antibody titers will increase. The inventors will then co-administer antigen@ZIF@polymer (the delayed release formulation) along with antigen@ZIF (FIG. 11) to create a continuous release. It is the inventors' working hypothesis that, even if the amount of protein is kept constant, this prolonged release will result in superior antibody titers as compared to a single injection of antigen.

Experimental Approach:

Principle experiment. Ovalbumin, CRM198, CPMV and Qβ @ZIF@polymer formulations that produced the strongest in vitro responses will be used. Each formulation will be tested individually. For each formulation, BALB/c mice aged 6-8 weeks will be divided into three separate groups of 12 (6M and 6F) and administered 10 μg of protein as either un-encapsulated antigen, antigen@ZIF, or antigen@ZIF@Polymer. Particle size and composition will be determined based on results from the previous aim. Mice will receive a single injection with no follow up booster shots.

Mice will be bled biweekly for serum samples and euthanized by week 11. Blood will be centrifuged, and serum tested by ELISA in various dilutions and titer will be calculated as the inverse dilution at which the sample matched the average absorbance. Ex vivo splenocytes cell suspensions will be prepared from spleens of sacrificed animals. Red blood cells will be lysed and the remaining splenocytes washed and isolated. Cells will be stimulated with the corresponding antigen in complete media. The supernatant will be collected and cytokine levels (outlined above) will be determined by ELISA. This experiment will be repeated by injecting a 1:1 mixture by protein weight of antigen@ZIF and antigen@ZIF@Polymer.

In vivo imaging of release. In a separate experiment, the inventors monitor protein release by using Cy5 or Cy7 labeled antigens. They have previously shown that dye labeling does not affect ZIF formation. The inventors will follow the procedure they developed previously with TMV@ZIF. Six mice (3F and 3M) will be subcutaneously injected with 10 μg Cy5-antigen@ZIF@polymer in the flank and imaged every 4-12 hours until fluorescence returns to baseline.

Data Analysis Plan: Sample size estimation. The inventors anticipate the variation in composition be minimal <5%. The inventors expect that, for the novel antigens@ZIF@polymer there will be larger variances in IgG and cytokine expression, which they expect to be around 20%. To detect at least a 20% difference in the protein expression changes with a power=0.8 and a significance level of 0.05, a sample size of at least 6 mice will be necessary for each sex (12 mice per group).

Controls. In addition to injection of the un-encapsulated antigen (detailed above) and protein free-ZIF, controls will include injection with just PBS, which will be a negative control. Subcutaneous injection with protein free ZIF@polymer into one leg and subcutaneous injection of the antigen in the other leg should produce the same response as injection of just the antigen. The inventors' “booster control group” will consist of a separate cohort of mice administered unencapsulated antigen on day 0 and given a follow up injection on day 7. All control groups will be treated the same as the experimental cohort. All samples will be administered blind to reduce experimentalist bias.

Expected Outcomes. When only antigen@ZIF@polymer is administered, the inventors anticipate seeing titers appear after day 10 and increase as the antigen is begins releasing from the freed antigen@ZIF. When antigen@ZIF and antigen@ZIF@polymer are co-administered, the inventors anticipate that IgG titers and cytokine levels will rise after 24 hours and consistently remain higher for longer as compared to the single injection group. In other words, IgG titers and cytokines will decline in single bolus groups while remaining steady in the “booster groups”. Further, the inventors anticipate a superior splenocyte response upon challenge of the antigen in the delayed release group. The single booster shot described in the booster control groups will provide improved IgG titers for the booster group as compared to the single injection group and the inventors will consider this aim a success if co-administration of antigen@ZIF and antigen@ZIF@polymer or exceeds this booster control group.

Alternative Strategies. Subcutaneous administration of the nanoparticles should remain in the interstitial space without diffusing or being distributed systematically. In mice that show no or weak responses to the subcutaneous administration of the antigen@ZIF@polymer the inventors will perform a paw pad drainage assay to verify the antigens are being taken in and delivered to the lymphatic system. Briefly, a small bolus of Cy5-antigen@ZIF@polymer is injected into the paw pad of a mouse where inflammation and drainage of particulates will occur into the lymph located in the armpit of the mouse. This lymph node can be excised and should show fluorescence from the accumulated antigen. If it does not, the nanoparticle formulations may be entering the blood stream from either poor injection technique or because the nanoparticles are too small, in which case, larger nanoparticles can be used.

In summary, the inventors will have formulated a high-yielding method to create long-lasting vaccine formulations that can be stored at room temperature and administered in a single shot.

### Example 3—PLGA Encapsulated ZIF-8 Nanoparticles: Sustained Release Vaccine Preparation

### A. Materials

ZIF-8 preparation: ZIF-8 was prepared using previous protocol where 20 mM 18.4 μL 2-methyl imidazole (HMIM) was reacted with 1200 mM 476.5 μL Zn Acetate and 505.1 μL water in each Eppendorf tube. It was kept at room temperature for 24 hours and washed with water and methanol and dried finally to get the Z1F-8 powders. SEM images were subsequently obtained (FIGS. 2A & 2B). The EHT in SEM was 2.4 kV. The size range of ZIF-8 was 389.3 nm to 795.5 nm.

Cy5@ZIF-8 preparation: In order to characterize the ZIF-8 nanoparticles in the blood the ZIF-8 particles were loaded with Cy5 dye (blue color) by following previous protocol. SEM images of the Cy5@ZIF-8 microparticles were subsequently obtained (FIGS. 3A & 3B).

Cy5@ZIF-8@PLGA microparticle preparation: A modified solid-in-oil-in-water (s/o/w) method was used to prepare the ZIF-8 microparticles. 0.05 g of ZIF 8 was taken and 0.1 g PEG-6000 was dissolved in water to obtain a concentration of 200 mg/mL. ZIF was then added to the solution in a 20:1 w/w PEG:ZIF-8 ratio. The total volume was 5 mL. The solution was sonicated for 20 mins to dissolve ZIF-8 and the mixture was kept in freezer overnight at −80° C. The sample was collected from freezer lyophilized then washed with dichloromethane to remove the PEG-6000. The sample was placed in Eppendorf tubes and centrifuged for 3225×g for 10 mins and the precipitate was collected. This process was repeated 3 times. The sample was then placed under vacuum for drying. S/O phase was prepared by suspending 20 mg of ZIF-8 microsphere powder in 2.5 mL DCM solution containing 125 mg PLGA.

PVA (2%) aqueous phase preparation: PVA wire (5 g) was taken and dissolved in enough water by boiling and stirring on a hot plate for 150° C. at 150 rpm for 2 hours to produce a 250 mL solution. 1% NaCl (2.5 g) was added to make 250 mL PVA aqueous solution then the oil phase was added to the 200 mL PVA solution and mixed with magnetic stirrer for 1.25 min at 340 rpm thereby producing the S/O/W emulsion.

Dilution: 1% NaCl containing 200 mL milliQ water was added and stirred with magnetic stir bar for 130 rpm for 12 hours.

### B. Characterization and Results

Scanning electron Microscopy: SEM images were obtained, and clear spherical particles were observed (FIGS. 4A & 4B). The sizes of particles ranged from 35 μm to 65.5 μm. To be sure that the spherical particles were ZIF-8 microspheres they were treated with chloroform and butanol to rupture the PLGA layer. SEM images were taken which ensured the presence of ZIF-8 inside microspheres (FIGS. 5A & 5B).

Fluorescence: The presence of Cy5 loaded ZIF-8 inside the PLGA microparticles was confirmed by fluorescence study. The Cy5 dye was synthesized in house. The emission and excitation wavelength of the Cy5 dye is 647 nm and 670 nm respectively. To validate the presence of Cy5 dye the fluorescence was done in water and methanol (FIGS. 6A & 6B).

PXRD: The presence of crystallinity of the Cy5@ZIF-8@PLGA microparticles was confirmed by powdered X ray diffraction analysis (FIGS. 7A & 7B). PLGA is a mixture of 50:50 crystal and amorphous character, which the PXRD data confirmed.

Ex vitro study: In order to observe the stability of PLGA microparticles in blood ex vitro study was done by the presence of fluorescence intensity after treating the Cy5@ZIF-8@PLGA particles in 1×PBS buffer. The experiment was performed at two different pH conditions. One was pH 7.4 which is the normal blood pH and another was pH 5.4 which mimic the extreme pH condition of blood in different disease states. The fluorescence intensity was measured at different time intervals. For that four types of samples were prepared every time. In two Eppendorf tubes PBS buffer of pH 7.4 and 5.4 were taken and fluorescence intensity was measured and found no peaks. PLGA microparticles were dissolved in 1×PBS buffer pH 7.4 and pH 5.4 with 10 mins centrifugation in Beckman ultra-small centrifuge machine at 3225×g. Only the dissolved clear supernatant was taken and kept in a hot room (37° C.). In another two Eppendorf tubes, Cy5 dye was dissolved in 1×PBS buffer pH 7.4 and 5.4 and kept under the same conditions as the PLGA microparticles to validate the change of fluorescence intensity of Cy5 dye at 37° C. (FIGS. 8A-8D).

Result and discussion: Incubation of Cy5 dye in 37° C. did not show any regular change of fluorescence intensity over time, whereas the PLGA microparticles showed decrease in fluorescence intensity up to 6 days in both pH conditions. It is believed that the PLGA microparticles further preserve the Cy5 dye and act as a sustained release protective shell which can enhance the release period of model drug Cy5 at blood pH.

Dynamic Light Scattering and Zeta potential:

| Sample | Zeta Potential (mV) |
|---|---|
| Cy5@ZIF-8 | −3.13 |
| Cy5@ZIF-8@PLGA | −5.103 |

### Example 4—Sustained Release Delivery of smURF Protein

### A. Experimental

Expression of smURFP: SmURF protein was expressed from E. coli by a heat shock method. The method is described on day basis. On the first day, yeast tryptone media was prepared. For each 1 L media, the recipe was tryptone (20 g), yeast extract (5 g), MgSO₄ (2.407 g), NaCl (0.5 g), and KCl (0.186 g). The media was autoclaved at P20 or P30 cycle. 50 mL/100 mL of previously autoclaved media was transferred in a 125 mL/250 mL erlenmeyer flask respectively. One single E. coli bacterial colony was selected and added to 50 mL culture media and was shaken at 37° C. overnight. Starter culture was divided evenly between 1 L flasks and was shaken at 37° C. until optical density reached 0.8-0.9. To induce expression, 10 g arabinose powder per liter was added and shaken overnight. Bacteria was palleted at 10.5 K rpm at 4° C. for 10 mins and resuspended in pH 8 1×PBS buffer. Cells were lysed in a microfluidizer. Cells were again palleted at 10.5 K rpm for 30 mins

FPLC was run with imidazole PBS solvent system to purify the smURF protein. Two characteristic peaks at 260 and 280 nm indicated the purified smURFP. It was then dialyzed for 72 hours in a dialysis bag in a cold room (4° C.) to concentrate the smURFP. Finally, the sample was lyophilized to get pure intense blue colored smURF protein.

Preparation of smURFP@ZIF-8: Different concentrations of smURF protein (1 mg/mL, 0.2 mg/mL and 0.3 mg/mL) were employed in encapsulation protocols to encapsulate the protein into ZIF-8. A 40:640 ratio of zinc acetate and 2-methyl imidazole reacted with different amounts of smURFP protein and milliQ water resulted in smURFP encapsulated ZIF-8 powders.

Preparation of smURFP@ZIF-8@PLGA microparticles: A modified s/o/w method was used to prepare the ZIF-8 microparticles. 0.05 g of smURFP@ ZIF 8 powder was obtained. 0.1 g PEG-6000 was dissolved in water to get 200 mg/mL concentration. Then the ZIF was added to that by 1:20 w/w ZIF-8: PEG ratio. The total volume was 5 mL. The solution was sonicated for 20 mins to dissolve ZIF-8. It was kept in freezer for overnight at −80° C. The sample was collected from freezer and kept it for lyophilization. The sample was collected from the lyophilizer and washed with dichloromethane to remove the PEG-6000. It was taken in Eppendorf tubes and centrifuged for 8000 rpm for 10 mins and the precipitate was collected. This was done for 3 times. Then the sample was kept in vacuum evaporator for drying. S/O phase was prepared by suspending 20 mg of ZIF-8 microsphere powder in 2.5 mL DCM solution containing 125 mg PLGA.

PVA (2%) aqueous phase preparation: 5 gm PVA wire was taken and dissolved in enough water by boiling and stirring on a hot plate for 150° C., 150 rpm for 2 hours to get 250 mL solution. 1% NaCl (2.5 g) was added to make 250 mL PVA aqueous solution. Then the oil phase was added to the 200 mL PVA solution and mixed with magnetic stirrer for 1.25 min at 340 rpm. Thus S/O/W emulsion was prepared.

### B. Characterization

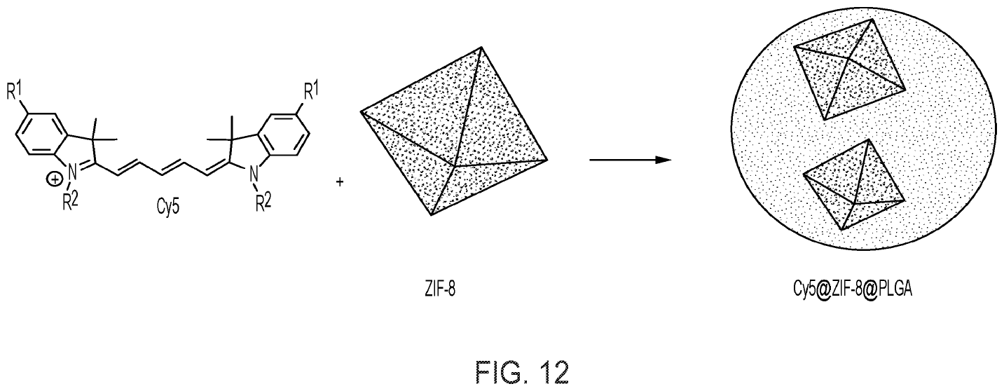

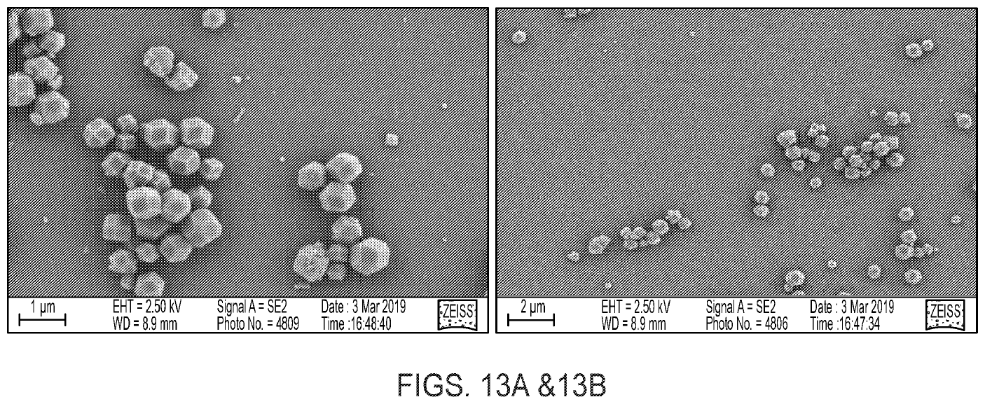

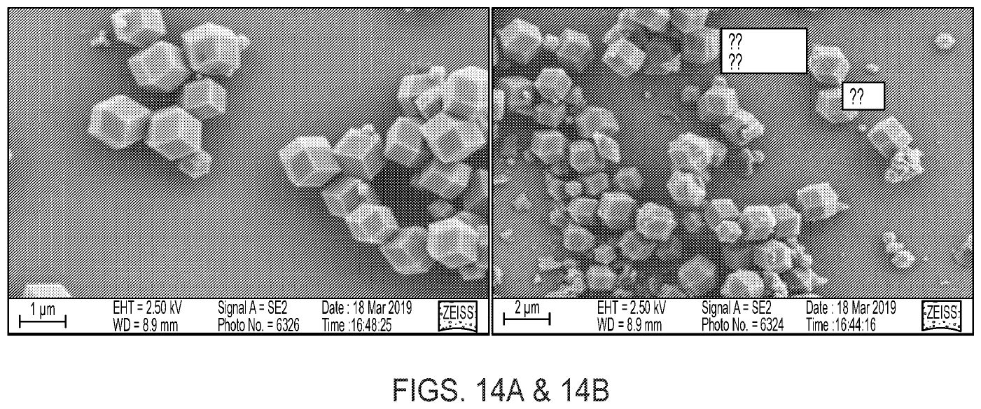

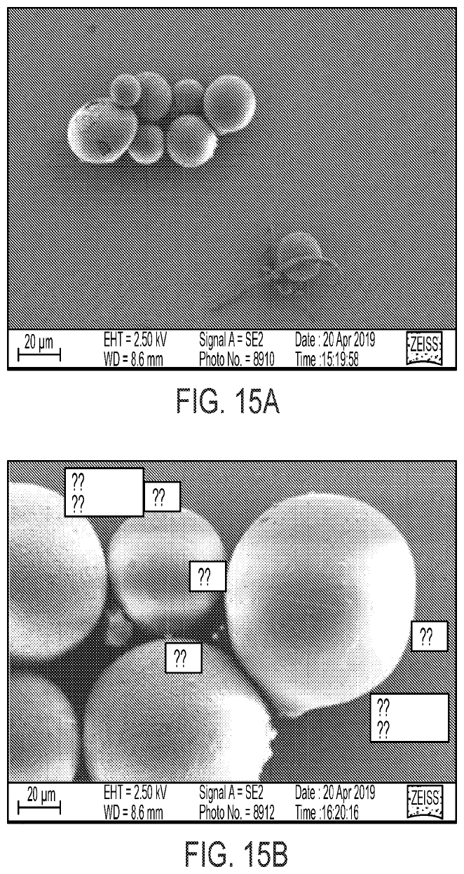

Scanning Electron Microscopy: SEM images of smURFP@ZIF-8 were obtained at various concentrations (FIGS. 11-13). SEM images of smURFP@ZIF-8@PLGA were also obtained (FIGS. 14A & 14B).

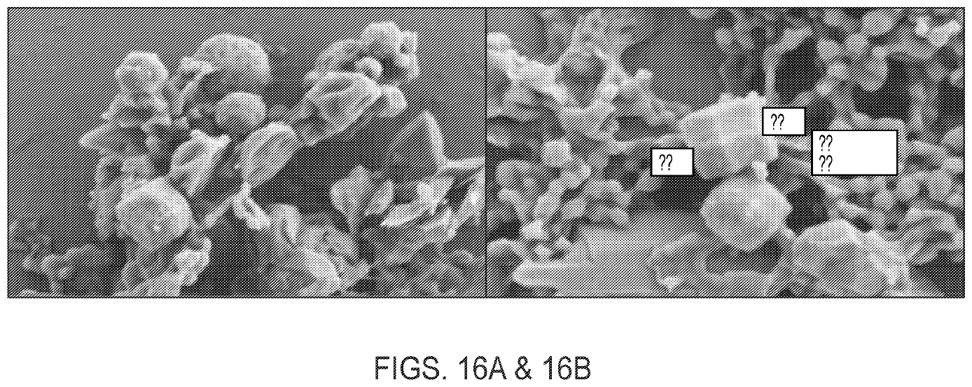

Fluorescence study: Solid and liquid state fluorescence data of smURFP@ZIF-8 and smURFP@ZIF@PLGA showed maximum emission at 670 nm after excitation at 642 nm which confirms presence of smURF protein in them (FIG. 16).

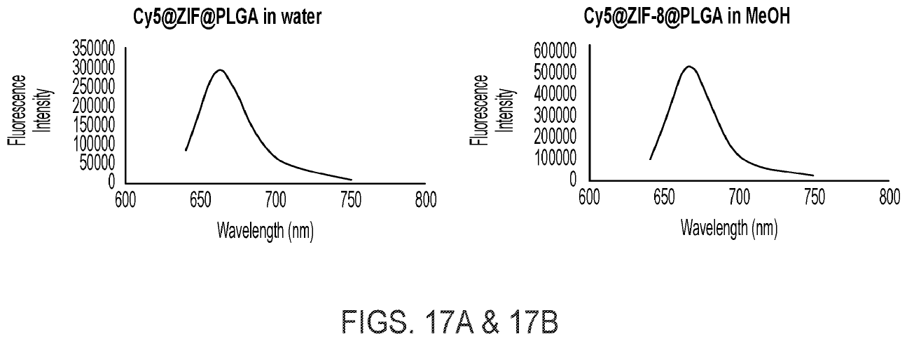

Gel Electrophoresis: smURFP@ZIF-8 particles were treated with exfoliation solution (0.5 M EDTA solution 600 pH 7.9) and then run through 1 Agarose gel to observe the presence of protein inside ZIF-8 molecules. Subsequently, the gel was imaged with Cy5 channel with Typhoon (FIG. 17A). The dark red bands indicated the presence of smURF proteins of different concentrations. The gel was also stained with comassie blue dye which also confirmed the protein inside the ZIF-8 (FIG. 17B).

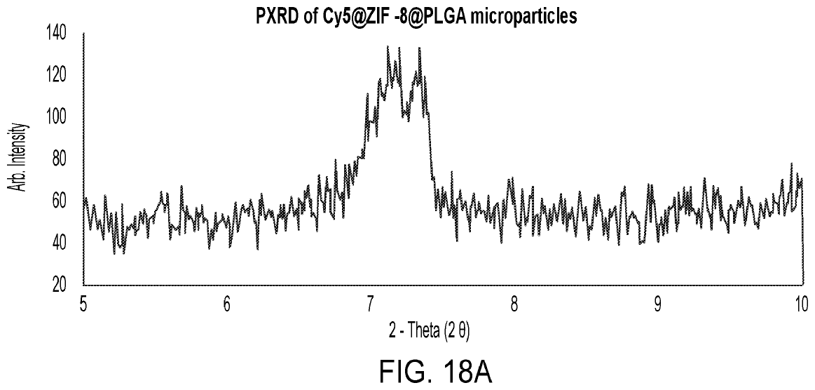

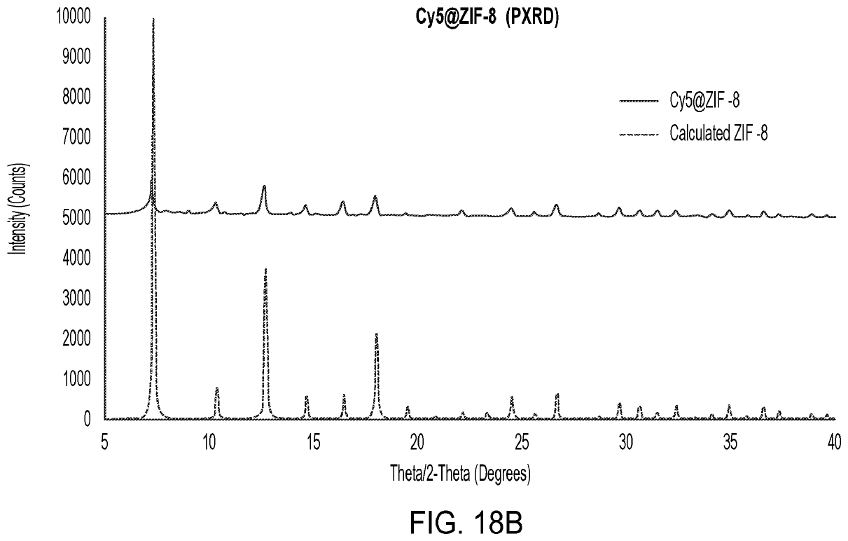

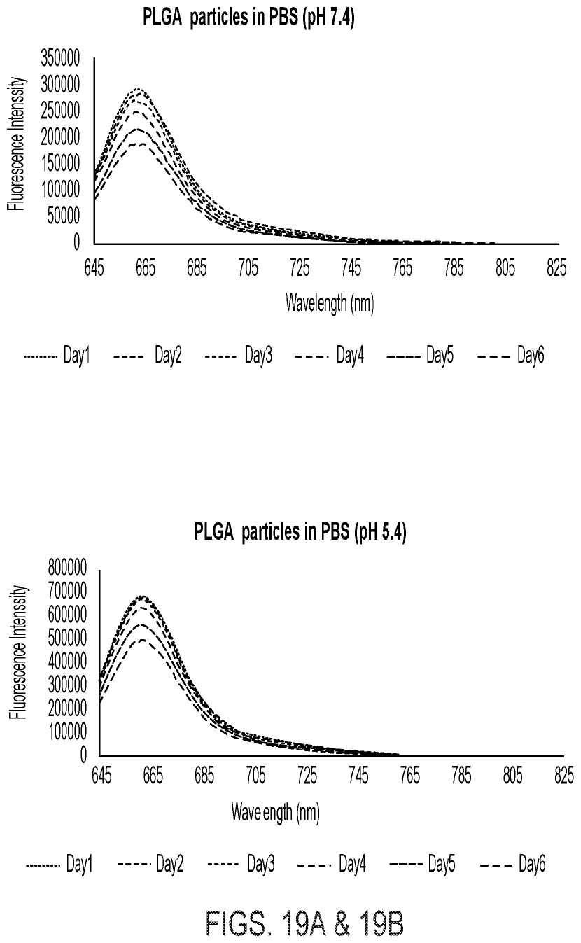

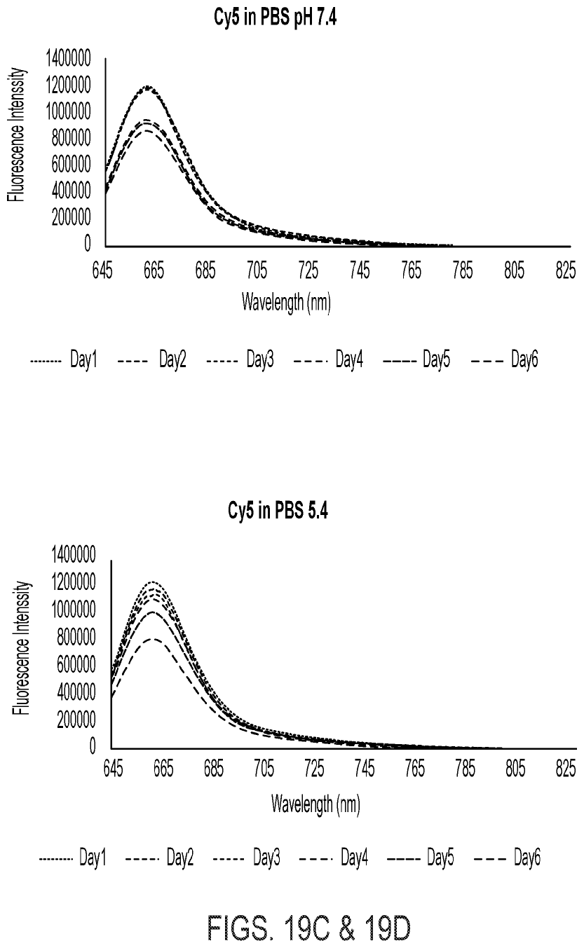

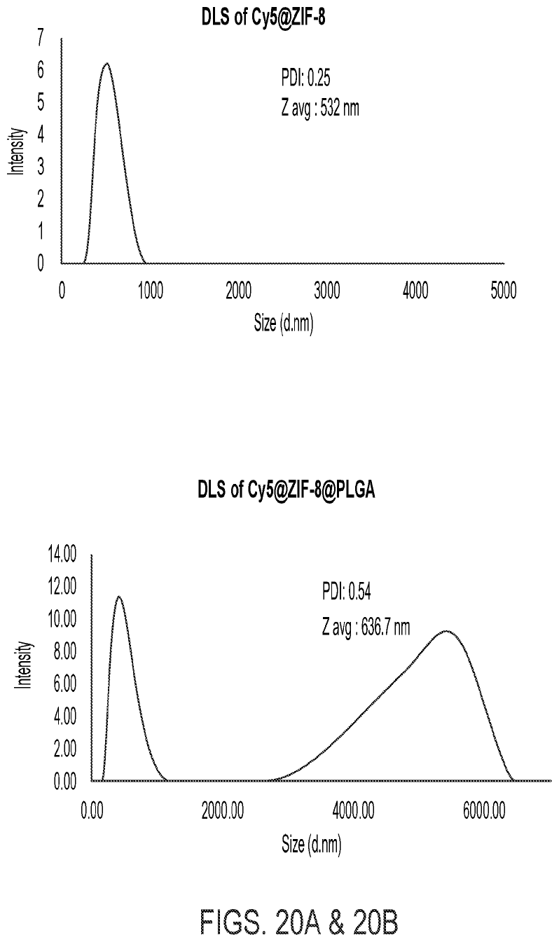

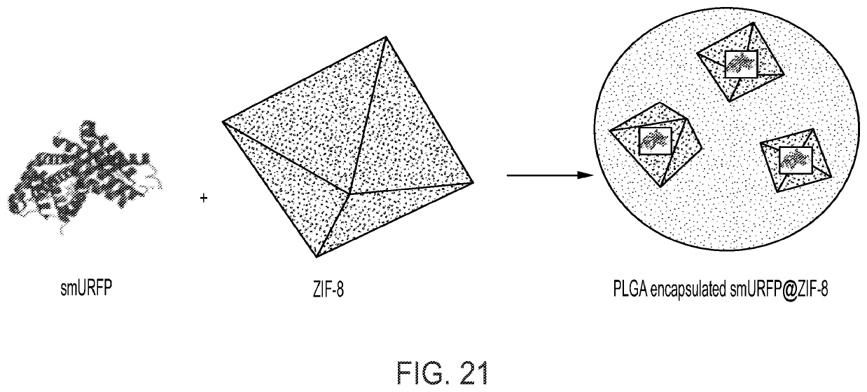

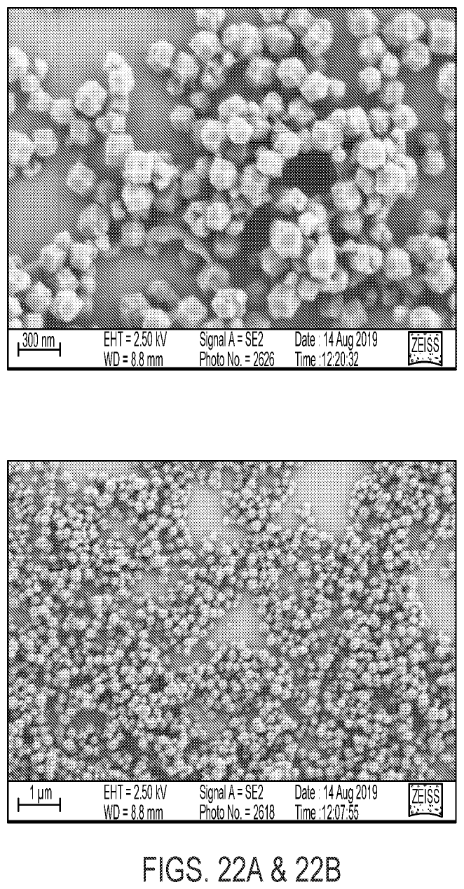

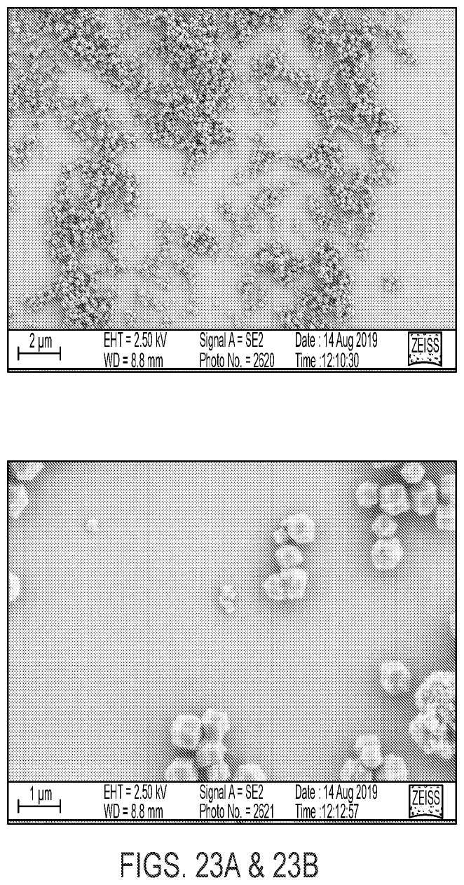

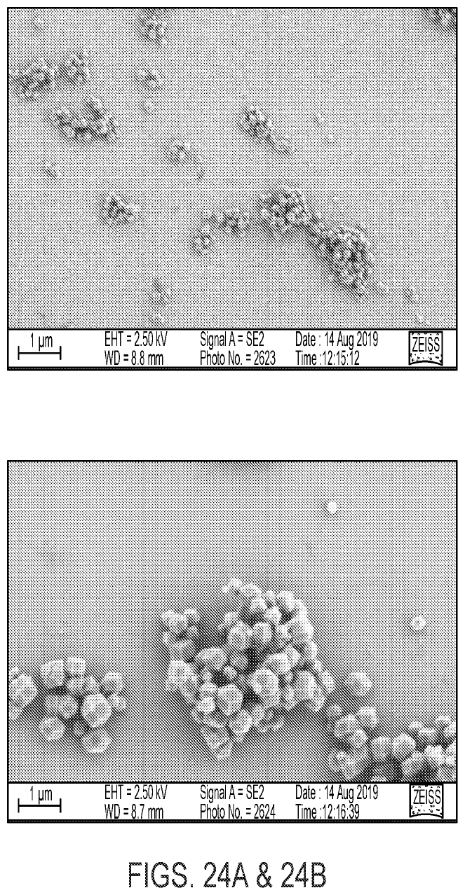

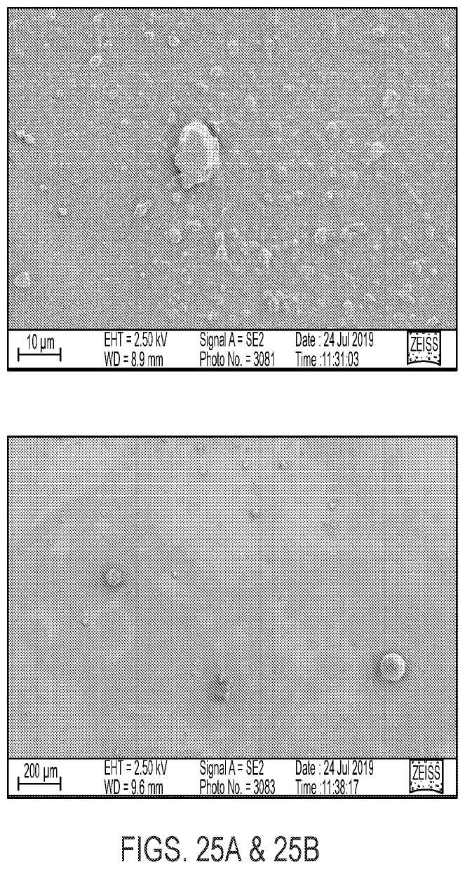

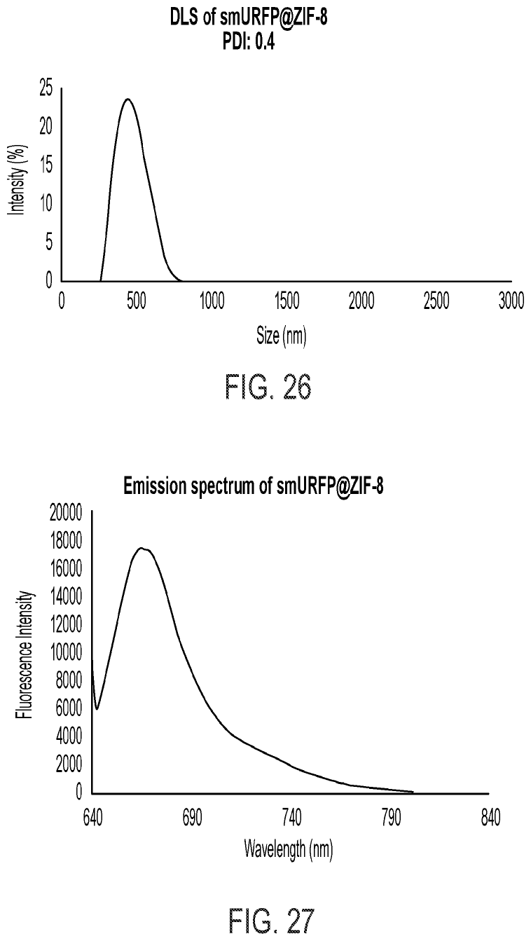

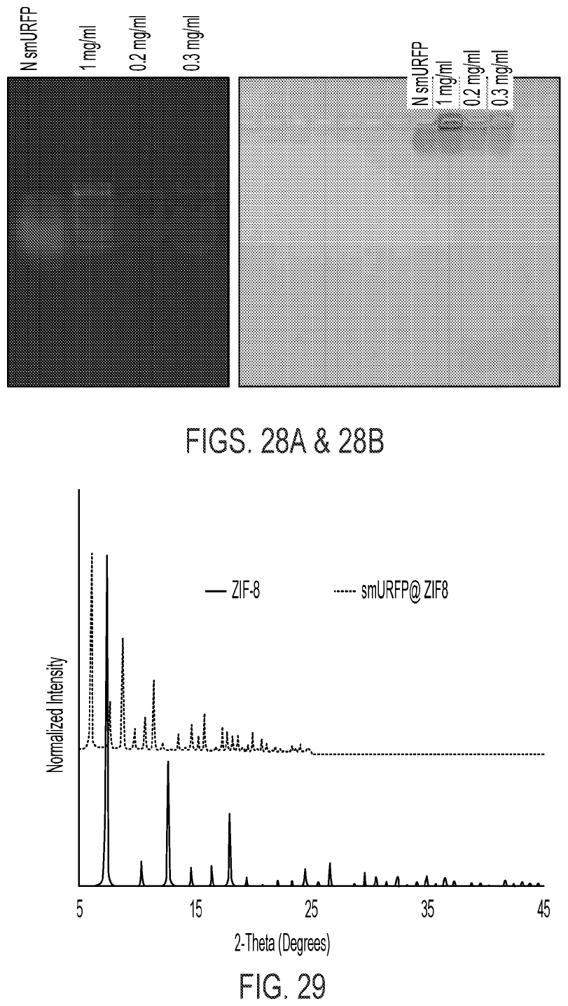

Powdered XRD: Powdered XRD data was taken with smart Rigaku XRD machine from 5 to 45 degrees, speed 3 (FIG. 18). PXRD data clearly shows similarity of XRD pattern of ZIF-8 and smURFP@ZIF-8 indicating that encapsulation of smURFP does not change the structural integrity of ZIF-8.

### Example 5—Additional Experiments

Modulation of the Size of the ZIF-8 Particles: The synthesis will be performed at room temperature and typically takes several minutes compared to hours and days in non-aqueous conditions. The obtained product ZIF-8 nanocrystals will have size of approximately 85 nm and will exhibit excellent thermal, hydrothermal and solvothermal stabilities. Tuning the stoichiometry and conditions during ZIF-8 formation will allow manipulation of the size of the resulting particles (Pan et al., 2011). With nano size ZIF-8 particles PLGA encapsulated ZIF-8 nanoparticles will be prepared instead of microspheres. This will help facilitate injection into a mouse model and provide enhanced bioavailability and kidney filtration. Further characterization of ZIF-8 and ZIF-8@PLGA nanoparticles will be performed to observe the size, shape, crystallinity and immune response after injecting into various mouse models.

In vivo study: 3 male mice groups will be selected and injected with 200 μl of ZIF-8, PLGA and ZIF-8@PLGA in each group. Then the skin of the mice will be shaved and the presence of Cy5 dye over time will be observed by animal imager by confirming the presence of fluorescence. Measurements will be performed to evaluate the time Cy5@ZIF-8@PLGA particles persist in the blood and the kinetics of release of the Cy5 drug model.

smURFP@ZIF-8@PLGA will be tested similarly. The ex vivo release of smURFP from PLGA microparticles can be monitored in different PBS solution @ 37° C. Fluorescence intensity will be measured each day to qualify and quantify the release of smURFP from PLGA particles.

All of the compositions and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compositions and methods without departing from the spirit, scope, and concept of the disclosure. All variations and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.

Anderson, Practical Process Research & Development—A Guide for Organic Chemists, 2n^{d} ed., Academic Press, New York, 2012.

Handbook of Pharmaceutical Salts: Properties, and Use, Stahl and Wermuth Eds., Verlag Helvetica Chimica Acta, 2002.

Reagan-Shaw et al., FASEB J., 22(3):659-661, 2008.

Smith, March's Advanced Organic Chemistry: Reactions, Mechanisms, and Structure, 7t^{h} Ed., Wiley, 2013.

Adhikari et al., Mol. Pharm., 12:3158-3166, 2015.

Ahn, RSC Adv., 5:15172-15181, 2015.

Alsaiari et al., J. Am. Chem. Soc., 140:143-146, 2018.

Anderson et al., Sci. Rep., 7:8431, 2017.

Backer et al., Sens. Actuators B, 238:716-722, 2017.

Banerjee et al., Science, 319:939-943, 2008.

Banik et al., PLoS One, 10:e0130858, 2015.

Carmichael and Shell, J. Chem. Phys., 143:243103, 2015.

Chen et al., ACS Appl. Mater. Interfaces, 10(3):2328-2337, 2018.

Chen et al., J. Am. Chem. Soc., 140:5678-5681, 2018.

Chen et al., J. Am. Chem. Soc., 140:9912-9920, 2018.

Chen et al., Nanomed. Nanobiotechnol., 8:512-534, 2016.

Cossé et al., AAPS PharmSciTech, 18:15-26, 2017.

Cui et al., Crystal Growth Des., 19(2), 1454-1470, 2019.

Deng et al., Science, 336:1018-1023, 2012.

Dharmarwardana et al., Mol. Pharmaceutics, 15:2973-2983, 2018.

Doonan et al., Acc. Chem. Res., 50:1423-1432, 2017.

Fan et al., ACS Sens., 3:441-450, 2018.

Finbloom et al., Bioconjugate Chem., 27:2480-2485, 2016.

Fuenzalida-Werner et al., J. Struct. Biol., 204(3):519-522, 2018.

Gasanova et al., Nanotechnol. Russ., 11:227-236, 2016.

Hayashi et al., Nat. Mater., 6:501-506, 2007.

He and Zhou, J. Microencapsul., 28:763-770, 2011.

Hoop et al., Appl. Mater. Today, 11:13-21, 2018.

Huxley et al., J. Am. Chem. Soc., 140:6416-6425, 2018.

Jha et al., Proc. Natl. Acad. Sci. U.S.A., 111:4856-4861, 2014.

Jun et al., Nat. Methods, 11(5):572-578, 2014.

Lázaro et al., ACS Appl. Mater. Interfaces, 10:31146-31157, 2018.

Leader et al., Nat. Rev. Drug Discov., 7:21-39, 2008.

LeClair et al., ACS Biomater. Sci. Eng., 4:1669-1678, 2018.

Lee et al., ACS Nano, 11:8777-8789, 2017.

Li et al., iACS Appl. Mater. Interfaces, 10(21):18161-18169, 2018.

Li et al., AIChE J., 64:3681-3689, 2018.

Li et al., Angew. Chem. Int. Ed., 55:10691-10696, 2016.

Li et al., Chem, 1:154-169, 2016.

Li et al., J. Am. Chem. Soc., 138:8052-8055, 2016.

Liang et al., Nat. Commun., 6:7240, 2015.

Liao et al., J. Am. Chem. Soc., 139:6530-6533, 2017.

Liu et al., Bioconjugate Chem., 28:836-845, 2017.

Luzuriaga et al., ACS Appl. Mater. Interfaces, 2019.

Lyu et al., Nano Lett., 14:5761-5765, 2014.

Maddigan et al., Chem. Sci., 9:4217-4223, 2018.

Majewski et al., CrystEngComm, 19:4082-4091, 2017.

Majewski et al., J. Mater. Chem. A, 6:7338-7350, 2018.

Mallamace et al., Proc. Natl. Acad. Sci. U.S.A., 113:3159-3163, 2016.

Masarapu et al., Biomacromolecules, 18:4141-4153, 2017.

Maurer et al., Eur. J. Immunol., 35:2031-2040, 2005.

McGuire and Forgan, Chem. Commun., 51:5199-5217, 2015.

Morales-Cruz, Results Pharm. Sci., 2:79-85, 2012.

Musselman, The University of Texas at Dallas, 2013.

Nadar et al., Enzyme Microb. Technol., 108:11-20, 2018.

Otake et al., J. Am. Chem. Soc., 140:8652-8656, 2018.

Pan et al., Chem. Commun., 47:2071-2073, 2011.

Park et al., Bioconjugate Chem., 23:350-62, 2012.

Pisal et al., AAPS PharmSciTech, 7:E30—E37, 2006.

Pitek et al., Mol. Pharmaceutics, 14:3815-3823, 2017.

Qi et al., ACS Sustainable Chem. Eng., 7(7):7127-7139, 2019.

Ren et al., Mater. Res. Bull., 48:4850-4855, 2013.

Riccò et al., ACS Nano, 12:13-23, 2018.

Rodriguez et al., Nat Methods, 13(9): 2016.

Rosi et al., CrystEngComm, 4:401-404, 2002.

Rybicki, Virol. J., 11:205, 2014.

Schade et al., Endocr. Pract., 23:1482-1484, 2017.

Shaner et al., Nat. Biotechnology, 22:1567, 2004.

Silva et al., ACS Omega, 3(9):12147-12157, 2018.

Sridhar et al., Biomacromolecules, 19:740-747, 2018.

Stack et al., Nat. Biotechnology, 18(12):1298-1302, 2000.

Stephanopoulos et al., Nat. Chem. Biol., 7:876-884, 2011.

Tanaka and Miyashita, ACS Omega, 2(10):6437-6445, 2017.

Teekampa, Int. J. Pharm., 534:229-234, 2017.

Thirumalai and Reddy, Nat. Chem., 3:910-911, 2011.

Vrdoljak et al., J. Controlled Release, 225:192-204, 2016.

Wang et al., ACS Appl. Mater. Interfaces, 8:26493-26500, 2016.

Wang et al., Adv. Healthcare Mater., 7:1800950, 2018.

Wang et al., Chem. Mater., 30:1291-1300, 2018.

Welch et al., Bioconjugate Chem., 29:2867-2883, 2018.

Wen et al., Bioconj. Chem., 30:515-524, 2019.

Xu et al., ACS Nano, 10:3267-3281, 2016.

Xu et al., J. Mater. Chem., 17:415-449, 2007.

Yandrapu and Upadhyay, Mol. Pharm., 10:4676-4686, 2013.

Zhang et al., ACS Appl. Mater. Interfaces, 9:31519-31525, 2017.

Zhang et al., Adv. Funct. Mater., 26:6454-6461, 2016.

Zheng et al., J. Am. Chem. Soc., 138:962-968, 2016.

Zhu et al., ACS Appl. Mater. Interfaces, 10:16066-16076, 2018.

Zhuang et al., ACS Nano, 8:2812-2819, 2014.

What is claimed:

1. A pharmaceutical composition comprising:
a) a therapeutic agent;
b) a metal-organic framework (MOF) or a coordination polymer; and
c) a pharmaceutically acceptable polymer;
wherein the therapeutic agent is encapsulated within the metal-organic framework or coordination polymer to form an encapsulated therapeutic agent, and wherein the encapsulated therapeutic agent is further encapsulated, entrapped, embedded, dispersed within, or complexed to the pharmaceutically acceptable polymer.

2. The composition of claim 1, wherein the metal-organic framework or coordination polymer comprises zirconium, iron, or zinc.

3. The composition of either claim 1 or claim 2, wherein the composition comprises a coordination polymer.

4. The composition of either claim 1 or claim 2, wherein the composition comprises a MOF.

5. The composition according to any one of claims 1, 2, and 4, wherein the metal-organic framework is a zeolitic imidazolate framework (ZIF).

6. The composition of claim 5, wherein the metal-organic framework is ZIF-8.

7. The composition according to any one of claims 1-6, wherein the therapeutic agent is a vaccine.

8. The composition according to any one of claims 1-6, wherein the therapeutic agent is a small molecule, a peptide or polypeptide, or a nucleotide or polynucleotide.

9. The composition of claim 8, wherein the therapeutic agent is a small molecule.

10. The composition of claim 9, wherein the small molecule is an antibiotic or a chemotherapeutic.

11. The composition of claim 8, wherein the therapeutic agent is a protein or a nucleic acid.

12. The composition according to any one of claims 1-6, wherein the therapeutic agent is derived from bacterial, protozoal, or microbial origin.

13. The composition according to any one of claims 1-6, wherein the therapeutic agent is a virus, a virus-like particle (VLP), a bacterium, or a bacterium-like particle (BLP).

14. The composition of claim 13, wherein the therapeutic agent is a virus.

15. The composition of claim 14, wherein the vaccine is an inactivated vaccine or a live-attenuated vaccine.

16. The composition according to any one of claims 1-6, wherein the therapeutic agent elicits an immune response.

17. The composition according to any one of claims 1-16, wherein the pharmaceutically acceptable polymer is polylactic acid.

18. The composition according to any one of claims 1-16, wherein the pharmaceutically acceptable polymer is polycaprolactone.

19. The composition according to any one of claims 1-16, wherein the pharmaceutically acceptable polymer is a co-polymer.

20. The composition of claim 16, wherein the co-polymer is a block co-polymer.

21. The composition according to any one of claims 1-20, wherein the pharmaceutically acceptable polymer is poly(lactic-co-glycolic acid).

22. The composition according to any one of claims 1-16, wherein the pharmaceutically acceptable polymer is a blend of polymers.

23. The composition of claim 22, wherein the blend comprises polylactic acid, polycaprolactone, or poly(lactic-co-glycolic acid).

24. The composition of claim 23, wherein the blend comprises polylactic acid, polycaprolactone, and poly(lactic-co-glycolic acid).

25. The composition according to any one of claims 1-24, wherein the composition is formulated as a colloid.

26. The composition according to any one of claims 1-25, wherein the composition is formulated for injection.

27. An implantable medical device comprising a composition according to any one of claims 1-25.

28. The device of claim 27, wherein the composition is comprised within a thin-film.

29. The device of claim 28, wherein the thin-film is present on the surface of the device.

30. A microneedle comprising a composition according to any one of claims 1-25.

31. The microneedle of claim 30, wherein the microneedle is coated with the composition.

32. The microneedle of claim 30, wherein the microneedle consists essentially of the composition.

33. The microneedle of claim 30, wherein the microneedle is attached to an adhesive patch.

34. A method of treating and/or preventing a disease or disorder in a subject in need thereof comprising administering to the subject an effective amount of a composition according to any one of claims 1-25.

35. The method of claim 34, wherein the composition comprises a vaccine.

36. A method of making a composition according to any one of claims 1-21 comprising contacting a therapeutic agent with a MOF and a pharmaceutically acceptable polymer.

37. The method of claim 36, wherein the method is performed in a single reaction vessel.

38. The method of claim 36, wherein the method further comprises a solvent.

39. The method of claim 38, wherein the solvent is water.

40. The method of claim 38, wherein the solvent is an aqueous solution comprising at least 50% water by volume.
